(19) United States
(12) Patent Application Publication
(10) Pub. No.: US 2024/0238267 A1
(43) Pub. Date: Jul. 18, 2024

(54) DRUG FORMULATIONS OF (R)-1-(1-ACRYLOYLPIPERIDIN-3-YL)-4-AMINO-3-(4-PHENOXYPHENYL)-1H-IMIDAZO[4,5-C]PYRIDIN-2(3H)-ONE

(71) Applicant: Genzyme Corporation, Cambridge, MA (US)

(72) Inventors: Marilyn Calderone, Chilly-Mazarin (FR); Caroline Viault, Chilly-Mazarin (FR)

(73) Assignee: Genzyme Corporation, Cambridge, MA (US)

(21) Appl. No.: 18/545,029

(22) Filed: Dec. 19, 2023

Related U.S. Application Data
(60) Provisional application No. 63/433,824, filed on Dec. 20, 2022.

(51) Int. Cl. A61K 31/4545; A61K 9/20; A61K 9/28

(52) U.S. Cl. CPC A61K 31/4545; A61K 9/2013; A61K 9/2018; A61K 9/2027; A61K 9/2054; A61K 9/284

(57) ABSTRACT

This disclosure relates to the field of therapeutic Bruton's tyrosine kinase (BTK) inhibitors. Pharmaceutical formulations of (R)-1-(1-acryloylpiperidin-3-yl)-4-amino-3-(4-phenoxyphenyl)-1H-imidazo[4,5-c]pyridin-2(3H)-one are described.

### CROSS-REFERENCE TO RELATED APPLICATIONS

This application claims priority to U.S. Provisional Application No. 63/433,824, filed on Dec. 20, 2022, which is incorporated by reference herein in its entirety for any purpose.

### FIELD OF THE DISCLOSURE

The present disclosure relates to pharmaceutical formulations of (R)-1-(1-acryloylpiperidin-3-yl)-4-amino-3-(4-phenoxyphenyl)-1H-imidazo[4,5-c]pyridin-2(3H)-one, the process of preparing the formulations, and methods of use thereof.

### BACKGROUND AND SUMMARY OF THE DISCLOSURE

BTK, a member of the Tec family non-receptor tyrosine kinases, is essential for B cell signaling downstream from the B-cell receptor. It is expressed in B cells and other hematopoietic cells such as monocytes, macrophages and mast cells. It functions in various aspects of B cell function that maintain the B cell repertoire (see Gauld S. B. et al., B cell antigen receptor signaling: roles in cell development and disease. Science, 296:1641-2. 2002.) B cells pay a role in rheumatoid arthritis (see Perosa F., et al., CD20-depleting therapy in autoimmune diseases: from basic research to the clinic. J Intern Med. 267:260-77. 2010 and Dörner T, et al. Targeting B cells in immune-mediated inflammatory disease: a comprehensive review of mechanisms of action and identification of biomarkers. Pharmacol Ther. 125:464-75. 2010 and Honigberg, L., et. al., The selective BTK inhibitor PCI-32765 blocks B cell and mast cell activation and prevents mouse collagen indiced arthritis. Clin. Immunol. 127 S1:S111. 2008) and in other autoimmune diseases such as systemic lupus erythematosus and cancers (see Shlomchik M. J., et. al., The role of B cells in lpr/lpr-induced autoimmunity. J. Exp Med. 180:1295-1306. 1994; Honigberg L. A., The Bruton tyrosine kinase inhibitor PCI-32765 blocks B-cell activation and is efficacious in models of autoimmune disease and B-cell malignancy. Proc. Natl. Acad. Sci. 107:13075-80. 2010; and Mina-Osorio P, et al., Suppression of glomerulonephritis in lupus-prone NZB×NZW mice by RN486, a selective inhibitor of Bruton's tyrosine kinase. Arthritis Rheum. 65: 2380-91. 2013).

There is also potential for BTK inhibitors for treating allergic diseases (see Honigberg, L., et. al., The selective BTK inhibitor PCI-32765 blocks B cell and mast cell activation and prevents mouse collagen indiced arthritis. Clin. Immunol. 127 S1:S111. 2008). It was noted that the irreversible inhibitor suppresses passive cutaneous anaphylaxis (PCA) induced by IgE antigen complex in mice. These findings agree with those noted with BTK-mutant mast cells and knockout mice and suggest that BTK inhibitors may be useful for the treatment of asthma, an IgE-dependent allergic disease of the airway.

The compound (R)-1-(1-acryloylpiperidin-3-yl)-4-amino-3-(4-phenoxyphenyl)-1H-imidazo[4,5-c]pyridin-2(3H)-one, (hereinafter also referred to as “Compound 1”), depicted below, is a BTK inhibitor:

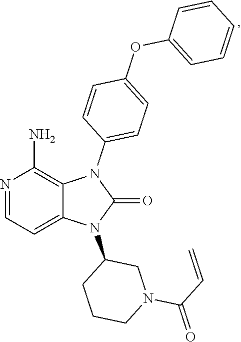 [SMILES: C=CC(=O)N1CCC[C@@H](n2c(=O)n(-c3ccc(Oc4ccccc4)cc3)c3c(N)nccc32)C1]

which is also known as “tolebrutinib,” and 4-amino-3-(4-phenoxyphenyl)-1-[(3R)-1-(prop-2-enoyl)piperidin-3-yl]-1,3-dihydro-2H-imidazo[4,5-c]pyridin-2-one having the following structure:

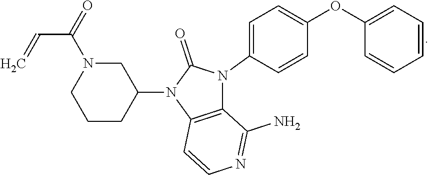 [SMILES: C=CC(=O)N1CCCC(n2c(=O)n(-c3ccc(Oc4ccccc4)cc3)c3c(N)nccc32)C1]

One factor in assessing the suitability of a compound as a therapeutic agent is whether the compound may be synthesized in a manner that is amenable to large scale manufacturing and isolation, with minimal product waste and impurities. This factor is frequently considered when reviewing the suitability of a bench-scale process for making the larger quantities needed for commercial production. For example, Compound (1) and a method for preparing it is disclosed in Example 3 of U.S. Pat. No. 9,688,676, herewith:

Into a 100-mL round-bottom flask, was placed (R)-4-amino-3-(4-phenoxyphenyl)-1-(piperidin-3-yl)-1H-imidazo[4,5-c]pyridin-2(3H)-one (150 mg, 0.37 mmol, 1.00 equiv), DCM-CH₃OH (6 mL), TEA (113 mg, 1.12 mmol, 3.00 equiv). This was followed by the addition of prop-2-enoyl chloride (40.1 mg, 0.44 mmol, 1.20 equiv) dropwise with stirring at 0° C. in 5 min. The resulting solution was stirred for 2 h at 0° C. The resulting mixture was concentrated under vacuum. The residue was applied onto a silica gel column with dichloromethane/methanol (30:1). The crude product (100 mg) was purified by Prep-HPLC with the following conditions (Column, XBridge Prep C18 OBD Column, 5 μm, 19*150 mm; mobile phase, water with 0.05% TFA and ACN (25.0% ACN up to 45.0% in 8 min). As noted above, this synthesis provides 100 mg of crude Compound (1) that must be purified by column chromatography, resulting in 54.5 mg of purified Compound (1), which can have a crystal Form 1 described hereinbelow.

Another, desirable aspect to be achieved is that the compound as a therapeutic agent can be administered in a form that is easily absorbed by the body and also shelf-stable. The pharmaceutically active substance used to prepare the treatment should be as pure as possible and its stability on long-term storage should be guaranteed under various environmental conditions. These properties are useful to prevent the appearance of unintended degradation products in pharmaceutical compositions, which degradation products may be potentially toxic or result simply in reducing the potency of the composition.

A primary concern for the large-scale manufacture of pharmaceutical compounds is that the active substance should have a stable crystalline morphology to ensure consistent processing parameters and pharmaceutical quality. If an unstable crystalline form is used, crystal morphology may change during manufacture and/or storage, resulting in quality control problems and formulation irregularities. Such a change may affect the reproducibility of the manufacturing process and thus lead to final formulations which do not meet the high quality and stringent requirements imposed on formulations of pharmaceutical compositions. In this regard, it should be generally borne in mind that any change to the solid state of a pharmaceutical composition which can improve its physical and chemical stability gives a significant advantage over less stable forms of the same drug.

When a compound crystallizes from a solution or slurry, it may crystallize with different spatial lattice arrangements, a property referred to as “polymorphism.” Each of the crystal forms is a “polymorph.” Although polymorphs of a given substance have the same chemical composition, they may differ from each other with respect to one or more physical properties, such as solubility, dissociation, true density, dissolution, melting point, crystal shape, compaction behavior, flow properties, and/or solid state stability.

Accordingly, the present disclosure seeks to address the deficiencies of the art. Disclosed herein are tablets comprising at least one compound chosen from (R)-1-(1-acryloylpiperidin-3-yl)-4-amino-3-(4-phenoxyphenyl)-1H-imidazo[4,5-c]pyridin-2(3H)-one (Compound 1) and pharmaceutically acceptable salts thereof; at least one diluent; at least one binder; at least one disintegrant; and at least one lubricant.

Also disclosed herein are methods of treating a disease or condition mediated by BTK in a patient in need thereof, comprising administering to the patient a tablet comprising at least one compound chosen from (R)-1-(1-acryloylpiperidin-3-yl)-4-amino-3-(4-phenoxyphenyl)-1H-imidazo[4,5-c]pyridin-2(3H)-one (Compound 1).

Also disclosed herein are uses of drug delivery forms, such as tablets, comprising at least one compound chosen from (R)-1-(1-acryloylpiperidin-3-yl)-4-amino-3-(4-phenoxyphenyl)-1H-imidazo[4,5-c]pyridin-2(3H)-one (Compound 1) for treating a disease involving mediation of the BTK receptor.

### BRIEF DESCRIPTION OF DRAWINGS

FIG. 1 shows a representation of torque as a function of water quantity/mixing weight.

FIG. 2 shows a representation of granulation curves with torque as a function of granulation time.

FIG. 3 shows a comparison of wetting curves for a Placebo Batch 1 and Formula A Batches 1 and 2.

FIG. 4 shows a comparison of wetting curves for Placebo Batches 1 and 2 and Formula A Batch 3.

FIG. 5 shows a comparison of wetting curves for Formula A Batches 1, 2, and 3.

FIG. 6 shows wetting curves for Formula B Batch 2.

FIG. 7 shows wetting curves and granulation at different percentages of water addition for Formula B Batch 2.

FIG. 8 shows a comparison of wetting curves and granulation curves for Formula B Batches 1 and 2.

FIGS. 9A, 9B, and 9C show comparative representations of wetting curves and granulation curves for Formula B Batch 2 and Formula C Batch 2.

FIG. 10 shows comparative representations of wetting curves and granulation curves for Formula C Batches 1 and 2.

FIG. 11 shows wetting curves and granulation of Formula E Batch 1, Formula C Batch 3, Formula D Batch 1, and Formula E Batch 2.

FIGS. 12A, 12B, and 12C show representations of particle size distribution, percent as a function of sieves for Formula E Batch 1, Formula C Batch 3, and Formula E Batch 2, respectively.

FIG. 13 shows a spider-like representation of Formula E Batch 1, Formula C Batch 3, and Formula D Batch 1.

FIG. 14 shows a hardness curve and disintegration time as a function of time for the 10 mg dosage strength tablet of Formula E Batch 2.

FIG. 15 shows a hardness curve and disintegration time as a function of time for the 60 mg dosage strength tablet of Formula E Batch 2.

FIG. 16 shows a comparison of 60 mg dosage strength core tablets for Formula E Batch 1, Formula C Batch 3, and Formula D Batch 1.

FIG. 17 shows a comparison of core and film-coated 10 mg dosage strength tablets of Formula E Batch 1.

FIG. 18 shows a comparison of core and film-coated 60 mg dosage strength tablets of Formula E Batch 1.

### DETAILED DESCRIPTION

Reference will now be made in detail to certain embodiments, examples of which are illustrated in the accompanying drawings. While the disclosure provides illustrated embodiments, it will be understood that they are not intended to limit the invention to those embodiments. On the contrary, the disclosure is intended to cover all alternatives, modifications, and equivalents, which may be included within the disclosure as defined by the appended claims.

The section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless otherwise stated, the following terms used in the specification and claims are defined for the purposes of this disclosure and have the following meaning:

The articles “a” and “an” are used in this disclosure to refer to one or more than one (i.e., to at least one) of the grammatical object of the article. By way of example, “an element” means one element or more than one element.

The term “about” is used in this disclosure to indicate and encompass an indicated value and a range above and below that value. In certain embodiments, the term “about” indicates the designated value ±10%, ±5%, or ±1%. In certain embodiments, where applicable, the term “about” indicates the designated value(s)±one standard deviation of that value(s).

The term “and/or” is used in this disclosure to mean either “and” or “or” unless indicated otherwise.

The terms “article of manufacture” and “kit” are used as synonyms.

As used herein, “the BTK inhibitor,” “the BTK inhibitor compound,” “the compound of Formula (1),” “Compound (1),” “tolebrutinib,” and “the compound,” refers to (R)-1-(1-acryloylpiperidin-3-yl)-4-amino-3-(4-phenoxyphenyl)-1H-imidazo[4,5-c]pyridin-2(3H)-one, having the following structure:

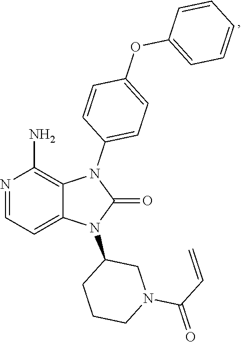 [SMILES: C=CC(=O)N1CCC[C@@H](n2c(=O)n(-c3ccc(Oc4ccccc4)cc3)c3c(N)nccc32)C1]

which is also known as 4-amino-3-(4-phenoxyphenyl)-1-[(3R)-1-(prop-2-enoyl)piperidin-3-yl]-1,3-dihydro-2H-imidazo[4,5-c]pyridin-2-one having the following structure:

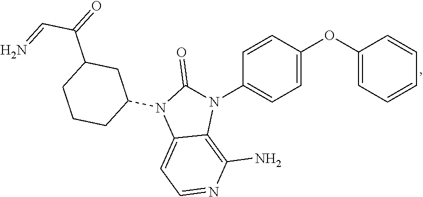

and/or a pharmaceutically acceptable salt thereof.

A “pharmaceutically acceptable carrier” or a “pharmaceutically acceptable excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

As used herein, the term “crystalline” or “crystalline solid form,” refers to a solid form which is substantially free of any amorphous solid-state form.

In some embodiments, “substantially free” means less than about 10% w/w, less than about 9% w/w, less than about 8% w/w, less than about 7% w/w, less than about 6% w/w, less than about 5% w/w, less than about 4% w/w, less than about 3% w/w, less than about 2.5% w/w, less than about 2% w/w, less than about 1.5% w/w, less than about 1% w/w, less than about 0.75% w/w, less than about 0.50% w/w, less than about 0.25% w/w, less than about 0.10% w/w, or less than about 0.05% w/w of other crystalline forms of the compound and the amorphous compound. In some embodiments, “substantially free” means an undetectable amount of other crystalline forms of the compound and the amorphous compound.

As used herein, the term “substantially pure” or “substantially crystalline” means that the crystalline form contains at least 90 percent, for example at least 95 percent, such as at least 97 percent, and even at least 99 percent by weight of the indicated crystalline form compared to the total weight of the compound of all forms.

Alternatively, it will be understood that “substantially pure” or “substantially crystalline” means that the crystalline form contains less than 10 percent, for example less than 5 percent, such as less than 3 percent, and even less than 1 percent by weight of impurities, including other polymorphic, solvated or amorphous forms compared to the total weight of the compound of all forms.

As used herein, the term “amorphous” refers to a solid material having no long-range order in the position of its molecules. Amorphous solids are generally supercooled liquids in which the molecules are arranged in a random manner so that there is no well-defined arrangement, e.g., molecular packing, and no long-range order. For example, an amorphous material is a solid material having no sharp characteristic signal(s) in its X-ray power diffractogram (i.e., is not crystalline as determined by XRPD). Instead, one or more broad peaks (e.g., halos) appear in its diffractogram. Broad peaks are characteristic of an amorphous solid.

As used herein, the term “substantially amorphous” refers to a solid material having little or no long-range order in the position of its molecules. For example, substantially amorphous materials have less than 15% crystallinity (e.g., less than 10% crystallinity or less than 5% crystallinity). “Substantially amorphous” includes the descriptor “amorphous,” which refers to materials having no (0%) crystallinity.

“Treating” or “treatment” of a disease includes:
(1) preventing the disease, e.g., causing the clinical symptoms of the disease not to develop in a mammal that may be exposed to or predisposed to the disease but does not yet experience or display symptoms of the disease;
(2) inhibiting the disease, e.g., arresting or reducing the development of the disease or its clinical symptoms; or
(3) relieving the disease, e.g., causing regression of the disease or its clinical symptoms.

“Optional” or “optionally” means that the subsequently described event or circumstance may but need not occur, and that the description includes instances where the event or circumstance occurs and instances in which it does not.

A “therapeutically effective amount” means the amount of the BTK inhibitor compound, that, when administered to a mammal for treating a disease, is sufficient to effect such treatment for the disease. The “therapeutically effective amount” will vary depending on the compound, the disease and its severity and the age, weight, etc., of the mammal to be treated.

Before describing the present teachings in detail, it is to be understood that the disclosure is not limited to specific compositions or process steps, as such may vary.

It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims.)

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “and/or,” unless the context requires otherwise.

### II. BTK inhibitor Compound

The BTK inhibitor compound can be prepared according to the methods and schemes described in, e.g., U.S. Pat. No. 9,688,676 B2, in particular the content of column 62, line 8 to column 65 line 32, and column 67, line 28 to column 69, which is incorporated herein by reference.

The following preparation of the compound of (R)-1-(1-acryloylpiperidin-3-yl)-4-amino-3-(4-phenoxyphenyl)-1H-imidazo[4,5-c]pyridin-2(3H)-one, is given to enable those skilled in the art to prepare the BTK inhibitor compound. The synthetic route should not be considered as limiting the scope of the disclosure, but merely as being illustrative and representative thereof.

Exemplary synthesis of (R)-1-(1-acryloylpiperidin-3-yl)-4-amino-3-(4-phenoxyphenyl)-1H-imidazo[4,5-c]pyridin-2(3H)-one:

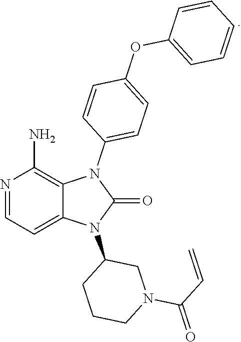 [SMILES: C=CC(=O)N1CCC[C@@H](n2c(=O)n(-c3ccc(Oc4ccccc4)cc3)c3c(N)nccc32)C1]

Into a 100 mL round-bottom flask was placed (R)-4-amino-3-(4-phenoxyphenyl)-1-(piperidin-3-yl)-1H-imidazo[4,5-c]pyridin-2(3H)-one (150 mg, 0.37 mmol, 1.00 equiv), DCM-CH₃OH (6 mL), TEA (113 mg, 1.12 mmol, 3.00 equiv). This was followed by the addition of prop-2-enoyl chloride (40.1 mg, 0.44 mmol, 1.20 equiv) dropwise with stirring at 0° C. in 5 min. The resulting solution was stirred for 2 h at 0° C. The resulting mixture was concentrated under vacuum. The residue was applied onto a silica gel column with dichloromethane/methanol (30:1). The crude product (100 mg) was purified by Prep-HPLC under the following conditions (Column, XBridge Prep C18 OBD Column, 5 μm, 19*150 mm; mobile phase, water with 0.05% TFA and ACN (25.0% ACN up to 45.0% in 8 min). 54.5 mg product of (R)-1-(1-acryloylpiperidin-3-yl)-4-amino-3-(4-phenoxyphenyl)-1H-imidazo[4,5-c]pyridin-2(3H)-one was obtained as a white solid. LC-MS m/z: 465.2 (M+1).

### III. Tablet Formulation

A tablet is provided that includes Compound 1 or a pharmaceutically acceptable salt thereof.

In some embodiments, a tablet is provided that comprises: at least one compound chosen from (R)-1-(1-acryloylpiperidin-3-yl)-4-amino-3-(4-phenoxyphenyl)-1H-imidazo[4,5-c]pyridin-2(3H)-one (Compound 1) and pharmaceutically acceptable salts thereof; at least one diluent; at least one binder; at least one disintegrant; and at least one lubricant.

In some embodiments, the tablet further comprises at least one flow agent.

In some embodiments, the tablet comprises an intragranular core and an extragranular portion.

In some embodiments, the at least one diluent is chosen from lactose monohydrate and microcrystalline cellulose. In some embodiments, the at least one diluent is present in a total amount ranging from about 70% to about 90% by weight. In some embodiments, the at least one diluent is present in the intragranular core in a total amount ranging from about 50% to about 70% by weight. In some embodiments, the at least one diluent is present in the extragranular portion in a total amount ranging from about 10% to about 30% by weight.

In some embodiments, the at least one binder is hydroxypropyl methylcellulose (hypromellose). In some embodiments, the at least one binder is present in a total amount ranging from about 0.1% to about 5% by weight.

In some embodiments, the at least one disintegrant is cross linked polyvinyl N-pyrrolidone (crospovidone). In some embodiments, the at least one disintegrant is present in a total amount ranging from about 0.1% to about 5% by weight. In some embodiments, the at least one disintegrant is present in the intragranular core in a total amount ranging from about 0.5% to about 3% by weight. In some embodiments, the at least one disintegrant is present in the extragranular portion in a total amount ranging from about 0.5% to about 3% by weight.

In some embodiments, the at least one lubricant is magnesium stearate. In some embodiments, the at least one lubricant is present in a total amount ranging from about 0.1% to about 5% by weight. In some embodiments, the at least one lubricant is present in a total amount of about 0.35% by weight.

In some embodiments, the at least one flow agent is talc. In some embodiments, the at least one flow agent is present in a total amount ranging from about 1% to about 5% by weight.

In some embodiments, the intragranular core comprises at least one diluent, at least one binder, and at least one disintegrant.

In some embodiments, the extragranular portion comprises at least one diluent, at least one disintegrant, and at least one lubricant.

In some embodiments, Compound 1 is present in at least 50% crystalline form, such as at least 60%, at least 70%, at least 80%, at least 90%, at least 95%, at least 96%, at least 97%, at least 98%, at least 99%, 99.5%, or 100% crystalline.

In some embodiments, Compound 1 is present in an amount ranging from about 0.9 mg to about 125 mg. In some embodiments, Compound 1 is present in an amount of about 1 mg, about 2.5 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, and about 125 mg. In some embodiments, Compound 1 is present in an amount of about 10 mg. In some embodiments, Compound 1 is present in an amount of about 60 mg. In some embodiments, Compound 1 is present in an amount of about 120 mg. In some embodiments, Compound 1 is present in an amount of about 5% to 25% by weight.

In some embodiments, the tablet further comprises at least one coating layer. In some embodiments, the coating layer comprises macrogol polyvinyl alcohol grafted copolymer, optionally further comprising talc with titanium dioxide. In some embodiments, the coating layer comprises hydroxypropyl methylcellulose. In some embodiments, the coating layer further comprises at least one of titanium dioxide and macrogol polyethylene glycol. In some embodiments, the coating layer further comprises at least one of copovidone and titanium dioxide.

In some embodiments, wherein the tablet comprises: about 15% to about 45% by weight lactose monohydrate, about 35% to about 60% by weight microcrystalline cellulose, about 1% to about 5% by weight hydroxypropyl methylcellulose, about 1% to about 5% by weight crospovidone, and about 0.1% to about 3% by weight magnesium stearate.

In some embodiments, tablet comprises an intragranular core and an extragranular portion. In some embodiments, the intragranular core comprises about 20% to about 40% by weight microcrystalline cellulose. In some embodiments, the extragranular portion comprises about 15% to about 25% by weight microcrystalline cellulose. In some embodiments, the intragranular core comprises about 0.5% to about 2.5% by weight crospovidone. In some embodiments, the extragranular portion comprises about 0.5% to about 2.5% by weight crospovidone. In some embodiments, Compound 1 is present in an amount of about 5% to 25% by weight.

In some embodiments, the tablet comprises: about 30% to about 40% by weight lactose monohydrate, about 40% to about 50% by weight microcrystalline cellulose, about 1% to about 3% by weight hydroxypropyl methylcellulose, about 2% to about 4% by weight crospovidone, and about 0.1% to about 1% by weight magnesium stearate.

In some embodiments, the tablet comprises an intragranular core and an extragranular portion. In some embodiments, the intragranular core comprises about 20% to about 30% by weight microcrystalline cellulose. In some embodiments, the extragranular portion comprises about 15% to about 25% by weight microcrystalline cellulose. In some embodiments, the intragranular core comprises about 0.5% to about 2.5% by weight crospovidone. In some embodiments, the extragranular portion comprises about 0.5% to about 2.5% by weight crospovidone. In some embodiments, Compound 1 is present in an amount of about 10% to 20% by weight.

In some embodiments, the tablet comprises: about 35.0% by weight lactose monohydrate, about 44.5% by weight microcrystalline cellulose, about 2.0% by weight hydroxypropyl methylcellulose, about 3.0% by weight crospovidone, and about 0.5% by weight magnesium stearate.

In some embodiments, the tablet comprises an intragranular core and an extragranular portion. In some embodiments, the intragranular core comprises 23.5% by weight microcrystalline cellulose. In some embodiments, the extragranular portion comprises 21.0% by weight microcrystalline cellulose. In some embodiments, the intragranular core comprises about 1.5% by weight crospovidone. In some embodiments, the extragranular portion comprises about 1.5% by weight crospovidone. In some embodiments, Compound 1 is present in an amount of about 15% by weight.

In some embodiments, the tablet comprises: about 28.8% by weight lactose monohydrate, about 55.8% by weight microcrystalline cellulose, about 2.0% by weight hydroxypropyl methylcellulose, about 3.0% by weight crospovidone, and about 0.4% by weight magnesium stearate.

In some embodiments, the tablet comprises an intragranular core and an extragranular portion. In some embodiments, the intragranular core comprises 34.8% by weight microcrystalline cellulose. In some embodiments, the extragranular portion comprises 21.0% by weight microcrystalline cellulose. In some embodiments, the intragranular core comprises about 1.5% by weight crospovidone. In some embodiments, the extragranular portion comprises about 1.5% by weight crospovidone. In some embodiments, Compound 1 is present in an amount of about 10% by weight.

In some embodiments, the tablet comprises: about 23.5% by weight lactose monohydrate, about 56.2% by weight microcrystalline cellulose, about 2.0% by weight hydroxypropyl methylcellulose, about 3.0% by weight crospovidone, and about 0.4% by weight magnesium stearate.

In some embodiments, the tablet comprises an intragranular core and an extragranular portion. In some embodiments, the intragranular core comprises 35.2% by weight microcrystalline cellulose. In some embodiments, the extragranular portion comprises 21.0% by weight microcrystalline cellulose. In some embodiments, the intragranular core comprises about 1.5% by weight crospovidone. In some embodiments, the extragranular portion comprises about 1.5% by weight crospovidone. In some embodiments, Compound 1 is present in an amount of about 15% by weight.

In some embodiments, the tablet comprises: about 35.2% by weight lactose monohydrate, about 44.5% by weight microcrystalline cellulose, about 2.0% by weight hydroxypropyl methylcellulose, about 3.0% by weight crospovidone, and about 0.4% by weight magnesium stearate.

In some embodiments, the tablet comprises an intragranular core and an extragranular portion. In some embodiments, the intragranular core comprises 23.5% by weight microcrystalline cellulose. In some embodiments, the extragranular portion comprises 21.0% by weight microcrystalline cellulose. In some embodiments, the intragranular core comprises about 1.5% by weight crospovidone. In some embodiments, the extragranular portion comprises about 1.5% by weight crospovidone. In some embodiments, Compound 1 is present in an amount of about 15% by weight.

In some embodiments, the tablet comprises: about 32.2% by weight lactose monohydrate, about 44.5% by weight microcrystalline cellulose, about 2.0% by weight hydroxypropyl methylcellulose, about 3.0% by weight crospovidone, about 0.4% by weight magnesium stearate; and about 3.0% by weight talc.

In some embodiments, the tablet comprises an intragranular core and an extragranular portion. In some embodiments, the intragranular core comprises 23.5% by weight microcrystalline cellulose. In some embodiments, the extragranular portion comprises 21.0% by weight microcrystalline cellulose. In some embodiments, the intragranular core comprises about 1.5% by weight crospovidone. In some embodiments, the extragranular portion comprises about 1.5% by weight crospovidone. In some embodiments, Compound 1 is present in an amount of about 15% by weight.

In some embodiments, the tablet further comprises a film-coating agent. In some embodiments, the film coating is present in an amount of about 2% to about 5% relative to the total weight of the tablet. In some embodiments, the film coating is present in an amount of about 3% relative to the total weight of the tablet.

In some embodiments, the ratio of lactose to intragranular microcrystalline cellulose is about 40:60 by weight. In some embodiments, the ratio of lactose to intragranular microcrystalline cellulose is about 50:50 by weight. In some embodiments, the ratio of lactose to intragranular microcrystalline cellulose is about 60:40 by weight.

In some embodiments, the coating layer comprises macrogol polyvinyl alcohol grafted copolymer, talc, titanium dioxide, glycerol monocaprylocaprate type 1/mono/diglycerides/glycerol of fatty acids, yellow ferric oxide, polyvinyl alcohol partly hydrolyzed, and red ferric oxide.

In some embodiments, the coating layer comprises hydroxypropyl methylcellulose, titanium dioxide, macrogol polyethylene glycol, yellow ferric oxide, and red ferric oxide.

In some embodiments, the coating layer comprises hydroxypropyl methylcellulose, copovidone, titanium dioxide, polyethylene glycol, yellow ferric oxide, indigo carmine aluminum lake, and caprylic/capric triglyceride.

The present disclosure also relates to a method of treating a disease or condition mediated by BTK in a patient in need thereof, comprising administering to the patient a tablet that comprises at least one compound chosen from (R)-1-(1-acryloylpiperidin-3-yl)-4-amino-3-(4-phenoxyphenyl)-1H-imidazo[4,5-c]pyridin-2(3H)-one (Compound 1) and pharmaceutically acceptable salts thereof; at least one diluent; at least one binder; at least one disintegrant; and at least one lubricant.

The present disclosure further relates to use of a tablet that comprises at least one compound chosen from (R)-1-(1-acryloylpiperidin-3-yl)-4-amino-3-(4-phenoxyphenyl)-1H-imidazo[4,5-c]pyridin-2(3H)-one (Compound 1) and pharmaceutically acceptable salts thereof; at least one diluent; at least one binder; at least one disintegrant; and at least one lubricant, for treating a disease involving mediation of the BTK receptor.

Diluents are chemical compounds that are used to dilute the compound of interest prior to delivery. Diluents can also be used to stabilize compounds because they can provide a more stable environment Salts dissolved in buffered solutions (which also can provide pH control or maintenance) are utilized as diluents in the art, including, but not limited to a phosphate buffered saline solution. In some embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pack (Amstar); hydroxypropyl-methylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some embodiments, the tablet includes at least one diluent chosen from mannitol, lactose monohydrate, anhydrous lactose, microcrystalline cellulose, starch, sorbitol, dextrose, dibasic calcium phosphate, dicalcium phosphate dihydrate, tricalcium phosphate, calcium phosphate, pregelatinized starch, compressible sugar, hydroxypropyl-methylcellulose, hydroxypropylmethylcellulose acetate stearate, sucrose-based diluents, confectioner's sugar, monobasic calcium sulfate monohydrate, calcium sulfate dihydrate, calcium lactate trihydrate, a dextrate, hydrolyzed cereal solids, amylose, powdered cellulose, calcium carbonate, glycine, kaolin, sodium chloride, inositol, and bentonite.

Binders impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinyl-pyrrolidone/vinyl acetate copolymer; crosspovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum mucilage of isapol husks, polyvinylpyrrolidone (e.g., Polyvidone® CL, Kollidon® CL, Polyplasdone® XL-10), larch arabogalactan, Veegum®, polyethylene glycol, polyethylene oxide, waxes, sodium alginate, and the like.

Those skilled in the art will appreciate that the selection of excipients may depend upon the process used to make the tablets before compression, whether that process involves blending, dry granulation or wet granulation. For example, dry granulation may involve compacting to form dry granules and then adding excipients later. Wet granulation may involve forming a weighting phase and granulation phase—with the particular excipients added to a specific phase—and then once the mixing of the phases has occurred, a drying occurs to obtain the granules. If wet granulation is chosen, the particular excipient may be selected to facilitate this process including the use of a binder. Of course, modifications of this may occur, such as adding materials later, as will be appreciated by skilled persons.

In some embodiments, the at least one binder is chosen from alginic acid, carboxymethylcellulose, methylcellulose (e.g., Methocel®), hydroxypropyl methylcellulose (Hypromellose, HPMC), hydroxyethylcellulose, hydroxypropylcellulose (e.g., Klucel®), ethylcellulose (e.g., Ethocel®), microcrystalline cellulose (e.g., Avicel®), microcrystalline dextrose, amylose, magnesium aluminum silicate, gelatin, polyvinyl-pyrrolidone/vinyl acetate copolymer; crosspovidone, povidone, starch, pregelatinized starch, tragacanth, dextrin, sucrose (e.g., Dipac®), glucose, dextrose, molasses, mannitol, sorbitol, xylitol (e.g., Xylitab®), lactose; acacia, tragacanth, ghatti gum mucilage of isapol husks, polyvinylpyrrolidone (e.g., Polyvidone® CL, Kollidon® CL, Polyplasdone® XL-10), larch arabogalactan, Veegum®, polyethylene glycol, polyethylene oxide, and sodium alginate.

Disintegrants contribute to both the dissolution and dispersion of the dosage form when contacted with gastrointestinal fluid. Disintegration agents or disintegrants facilitate the disintegration of a tablet and granules and consequently affect the release of drug substance. Examples of disintegration agents include a starch, e.g., a natural starch such as corn starch or potato starch, a pregelatinized starch such as National 1551 or sodium starch glycolate such as Promogel® or Explotab®, a cellulose such as a wood product, methylcrystalline cellulose, e.g., Avicel®, Avicel® PH101, Avicel® PH 102, Avicel® PH105, Elceme® P100, Emcocel®, Vivacel®, and Solka-Floc®, methylcellulose, croscarmellose, or a cross-linked cellulose, such as cross-linked sodium carboxymethyl-cellulose (Ac-Di-Sol®), cross-linked carboxymethylcellulose, or cross-linked croscarmellose, a cross-linked starch such as sodium starch glycolate, a cross-linked polymer such as cross linked polyvinyl N-pyrrolidone (crospovidone), a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some embodiments, the at least one disintegrant is chosen from sodium starch glycolate, croscarmellose sodium, corn starch, potato starch, pregelatinized starch, methylcrystalline cellulose, methylcellulose, croscarmellose, cross-linked sodium carboxymethyl-cellulose, cross-linked carboxymethylcellulose, cross-linked croscarmellose, crospovidone, cross-linked polyvinylpyrrolidone, alginic acid, sodium alginate, magnesium aluminum silicate, agar, guar, locust bean, Karaya, pectin, tragacanth, bentonite, citrus pulp, and sodium lauryl sulfate.

Lubricants are compounds that prevent, reduce, or inhibit adhesion or friction of materials. Exemplary lubricants include, e.g., stearic acid, calcium hydroxide, talc, sodium stearyl lumerate, a hydrocarbon such as mineral oil, or hydrogenated vegetable oil such as hydrogenated soybean oil, higher fatty acids and their alkali-metal and alkaline earth metal salts, such as aluminum, calcium, magnesium, zinc, stearic acid, sodium stearates, glycerol, talc, waxes, boric acid, sodium benzoate, sodium acetate, sodium chloride, leucine, a polyethylene glycol (e.g., PEG4000) or a methoxypolyethylene glycol such as Carbowax®, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

In some embodiments, the at least one lubricant is chosen from magnesium stearate, stearic acid, calcium hydroxide, talc, sodium stearyl lumerate, mineral oil, hydrogenated soybean oil, aluminum, calcium, magnesium, zinc, sodium stearate, glycerol, talc, wax, boric acid, sodium benzoate, sodium acetate, sodium chloride, leucine, polyethylene glycol, methoxypolyethylene glycol, sodium oleate, sodium benzoate, glyceryl behenate, magnesium lauryl sulfate, sodium lauryl sulfate, colloidal silica, corn starch, silicone oil, and surfactant.

In some embodiments, the tablet comprises at least one flow agent. In some embodiments, the flow agent is silica or talc.

In some embodiments, the tablet may include at least one pH adjusting agent and/or buffering agent, for example, acids such as acetic, citric, fumaric, maleic, tartaric, malic, lactic, phosphoric and hydrochloric acids; bases such as sodium hydroxide, sodium phosphate, sodium borate, sodium citrate, sodium lactate and tris-hydroxymethylaminomethane; and buffers such as citrate, sodium bicarbonate, ammonium chloride, and the like. Such buffers used as bases may have other counterions than sodium, for example, potassium, magnesium, calcium, ammonium, or other counterions. Such acids, bases and buffers are included in an amount required to maintain pH of the composition in an acceptable range.

In some embodiments, the tablet may also include at least one antifoaming agent to reduce foaming during processing which can result in coagulation of aqueous dispersions, bubbles in the finished film, or generally impair processing. Exemplary anti-foaming agents include silicon emulsions or sorbitan sesquoleate.

In some embodiments, the tablet may also include at least one salt in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some embodiments, the tablet may also include at least one antioxidant, such as non-thiol antioxidants, for example, butylated hydroxytoluene (BHT), sodium ascorbate, ascorbic acid or its derivative, and tocopherol or its derivatives. In certain embodiments, antioxidants enhance chemical stability where required. Other agents such as citric acid or citrate salts or EDTA may also be added to slow oxidation.

In some embodiments, the tablet may also include at least one preservative to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide, and cetylpyridinium chloride.

In some embodiments, the tablet may also include at least one dispersing agent and/or viscosity modulating agent. Dispersing agents and/or viscosity modulating agents include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween®60 or 80, polyvinylpyrrolidone (PVP; commercially known as Plasdone®), triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®., and F10®8, which are block copolymers of ethylene oxide and propylene oxide); and poloxamines (e.g., Tetronic 9088, also known as Poloxamine 908®, which is a tetrafonctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polysorbate-80, gums, such as, e.g., gum tragacanth and gum acacia, guar gum, xanthans, including xanthan gum, polyethoxylated sorbitan monolaurate, polyethoxylated sorbitan monolaurate, and carbomers. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate. In general, binder levels of about 10 to about 70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 90% and more typically up to 70% in tablet formulations is common.

In some embodiments, the tablet may also include at least one erosion facilitator. Erosion facilitators include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

In some embodiments, the tablet may also include at least one filling agent, which includes compounds such as dextrates, dextran, sucrose, xylitol, lactitol, and the like.

In some embodiments, the tablet may also include at least one flavoring agent and/or sweetener e.g., acacia syrup, acesulfame K, alitame, anise, apple, aspartame, banana, Bavarian cream berry, black currant, butterscotch, calcium citrate, camphor, caramel, cherry, cherry cream chocolate, cinnamon, bubble gum, citrus, citrus punch, citrus cream, cotton candy, cocoa, cola, cool cherry, cool citrus, cyclamate, cyclamate, eucalyptus, eugenol, fructose, fruit punch, ginger, glycyrrhizinate, glycyrrhiza (licorice) syrup, grape, grapefruit, honey, isomalt, lemon, lime, lemon cream, monoammonium glyrrhizinate, maltol, maple, marshmallow, menthol, mint cream, mixed berry, neohesperidine DC, neotame, orange, pear, peach, peppermint, peppermint cream, Powder, raspberry, root beer, rum, saccharin, safrole, spearmint, spearmint cream, strawberry, strawberry cream, stevia, sucralose, sucrose, sodium saccharin, saccharin, aspartame, acesulfame potassium, talin, xylitol, sucralose, Swiss cream, tagatose, tangerine, thaumatin, tutti frutti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

In some embodiments, the tablet may also include at least one solubilizer, which includes compounds such as triacetin, triethylcitrate, ethyl oleate, ethyl caprylate, sodium doccusate, vitamin E TPGS, dimethylacetamide, N-methylpyrrolidone, N-hydroxyethylpyrrolidone, hydroxypropyl cyclodextrins for example Captisol®, ethanol, n-butanol, isopropyl alcohol, cholesterol, bile salts, glycofurol, transcutol, and dimethyl isosorbide and the like. In one embodiment, the solubilizer is vitamin E TPGS and/or Captisol® or β-hydroxypropylcyclodextrin.

In some embodiments, the tablet may also include at least one suspending agent, which includes compounds such as vinyl pyrrolidone/vinyl acetate copolymer (S630), polysorbate-80, hydroxyethylcellulose, gums, such as, e.g., gum tragacanth and gum acacia, guar gum, xanthans, including xanthan gum, polyethoxylated sorbitan monolaurate, polyethoxylated sorbitan monoleate, and the like.

In some embodiments, the tablet may also include at least one surfactant, which includes compounds such as sodium docusate, Tween 20, 60 or 80, triacetin, vitamin E TPGS, sorbitan monooleate, polyoxyethylene sorbitan monooleate, polyoxyethylene sorbitan monolaurate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g. octoxynol 10, octoxynol 40. In some embodiments, surfactants may be included to enhance physical stability or for other purposes.

In some embodiments, the tablet may also include at least one wetting agent, which includes compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium doccusate, triacetin, Tween 80, vitamin E TPGS, ammonium salts and the like.

Pharmaceutical preparations disclosed herein can be obtained by mixing at least one solid excipient described herein, with Compound 1 described herein, optionally grinding the resulting mixture, and processing the mixture of granules, after adding suitable excipients, if desired, to obtain tablets.

Pharmaceutical preparations disclosed herein also include capsules made of gelatin, as well as soft, sealed capsules made of gelatin and a plasticizer, such as glycerol or sorbitol. Capsules may also be made of polymers such as Hypromellose (i.e., hydroxypropyl methylcellulose). The capsules can contain the active ingredients, optionally in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, lipids, solubilizers, or liquid polyethylene glycols. In addition, stabilizers may be added. All formulations for oral administration should be in dosages suitable for such administration.

These formulations can be manufactured by conventional pharmacological techniques. Conventional pharmacological techniques include, e.g., one or a combination of methods: (1) dry mixing, (2) direct compression, (3) milling, (4) dry or non-aqueous granulation, (5) wet granulation, (6) fusion, or (7) extrusion. See, e.g., Lachman et al., The Theory and Practice of Industrial Pharmacy, 3^{rd} ed. (1986). Other methods include, e.g., spray drying, pan coating, melt granulation, granulation, fluidized bed spray drying or coating (e.g., wurster coating), tangential coating, top spraying, tableting, extruding, extrusion/spheronization, and the like.

It should be appreciated that there is considerable overlap between excipients used in the solid dosage forms described herein. Thus, the above-listed additives should be taken as merely exemplary, and not limiting, of the types of excipient that can be included in solid dosage forms described herein. The type and amounts of such excipient can be readily determined by one skilled in the art, according to the particular properties desired.

Colorants, surfactants, anti-adhesion agents, antifoaming agents, lubricants (e.g., carnauba wax or PEG) and other additives may be added to the coatings besides plasticizers to solubilize or disperse the coating material, and to improve coating performance and the coated product.

In some embodiments, the tablets are packaged in a blister pack. The blister packs can be made from any appropriate material. Examples include polyvinyl-polychlorotrifluoroethylene in the forming film and hardened aluminum foil as lidding.

### IV. Liquid Formulation

A liquid suspension can be prepared by adding a tablet to water and crushing the tablet to disperse it in the water.

In some embodiments, a syringe can be provided that includes a liquid suspension of a crushed tablet. The syringe can be used to connect to a gastric tube and empty it into a gastric tube.

### V. Therapeutic Methods

Provided herein are methods of treating a disease or condition mediated by BTK comprising administering to a subject in need thereof a therapeutically effective amount of the BTK inhibitor compound comprising (R)-1-(1-acryloylpiperidin-3-yl)-4-amino-3-(4-phenoxyphenyl)-1H-imidazo[4,5-c]pyridin-2(3H)-one or a pharmaceutically acceptable salt thereof. In some embodiments the therapeutically effective amount is about 1 to about 125 mg. In some embodiments, the subject is a mammal. In some embodiments, the mammal is a human. In some embodiments, the subject has one or more symptoms of (i) multiple sclerosis (MS), including relapsing forms of MS, non-relapsing secondary progressive multiple sclerosis (NRSPMS), and primary progressive multiple sclerosis (PPMS); (ii) myasthenia gravis (MG); or (iii) Myelin oligodendrocyte glycoprotein antibody disease (MOGAD) prior to treatment and the treatment reduces or eliminates the one or more symptoms. In some embodiments, the subject suffers from inflammation, pain, loss of mobility, and muscular weakness caused by MS, MG or MOGAD.

In some embodiments, a subject with MS has at least one documented relapse within the previous year, and/or at least two documented relapses within the previous two years, and/or at least one active Gd-enhancing brain lesion on an MRI scan within the previous year and prior to screening.

In some embodiments, a dose of about 5-10 mg, 10-15 mg, 15-20 mg, 20-25 mg, 25-30 mg, 30-35 mg, 35-40 mg, 40-45 mg, 45-50 mg, 50-55 mg, 55-60 mg, 60-65 mg, 65-70 mg, 70-75 mg, 75-80 mg, 80-85 mg, 85-90 mg, 90-95 mg, 95-100 mg, 100-105 mg, 105-110 mg, 110-115 mg, 115-120 mg, or 120-125 mg is administered. In some embodiments, the dose is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, or about 125 mg. In some embodiments, the dose is about 5 mg. In some embodiments, the dose is about 10 mg. In some embodiments, the dose is about 20 mg. In some embodiments, the dose is about 30 mg. In some embodiments, the dose is about 60 mg. In some embodiments, the dose is about 120 mg.

In some embodiments, the dose is administered daily. The daily dose can be delivered as a single dose or split into multiple parts. For example, in some embodiments, the dose is administered once a day (e.g., about every 24 hours). In some embodiments, the dose is administered twice daily. In some embodiments, the dose is subdivided in two parts to be administered twice per day (e.g., about every 12 hours). In some embodiments, the dose is subdivided in three parts to be administered three times per day (e.g., about every 8 hours). In some embodiments, the dose is subdivided in four parts to be administered four times per day (e.g., about every 6 hours).

In some embodiments, the dose is administered orally. In some embodiments, the dose is administered in a form of tablets. In some embodiments, the dose is administered in the form of pills, capsules, semisolids, powders, sustained release formulations, solutions, suspensions, elixirs, aerosols, or any other appropriate compositions.

In some embodiments, the subject is administered the BTK inhibitor compound for a period of about 4, 8, 12, 16, 20, 24, or 26 weeks. In some embodiments, the subject is administered the BTK inhibitor compound for a period of about 6, 12, 18, 24, 30, 36, 42, or 48 months. In some embodiments, the dose is once daily.

In some embodiments, the BTK inhibitor compound is administered as monotherapy. In some embodiments, the method comprises administering the BTK inhibitor compound and at least one additional therapeutic agent. The additional therapeutic agent may be administered concurrently or sequentially with the BTK inhibitor compound.

Determination of the frequency of administration can be made by persons skilled in the art, such as an attending physician based on considerations of the condition being treated, age of the subject being treated, severity of the condition being treated, general state of health of the subject being treated and the like. In some embodiments, BTK inhibitor compounds are administered in a therapeutically effective amount for treatment of MS, MG, or MOGAD. The therapeutically effective amount is typically dependent on the weight of the subject being treated, his or her physical or health condition, the extensiveness of the condition to be treated, or the age of the subject being treated, pharmaceutical formulation methods, and/or administration methods (e.g., administration time and administration route).

The foregoing disclosure has been described in some detail by way of illustration and example, for purposes of clarity and understanding. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

### EXAMPLES

The following examples are provided to illustrate certain disclosed embodiments and are not to be construed as limiting the scope of this disclosure in any way. In the Examples discussed below, the BTK inhibitor, as defined above, may be also referred as “Compound 1”, “the compound,” “tolebrutinib,” or “the drug” interchangeably.

### Example 1: Preparation of Tablets Containing Compound 1

Development of the tablet formulations described in this example involved experiments to determine:
the concentration of the active ingredient that should be used,
whether to use hydroxypropyl methylcellulose (HPMC) in the dry mixing or in the aqueous binding solution,
the ratio of lactose to microcrystalline cellulose that should be used, and
the effect of the addition of silica or other test flow agent such as talc.

The process further involved adjusting:
Wetting time with an adaptation of the flow rate of the mooring solution
Volume of mooring water
Granulation time

Calibration of the dried granules in its entirety directly compared to the two steps on the Phase IIb formulation: sieving of the dry granules and then calibration of the unscrewed granules. The excipients used for this study are shown in Table 1.

TABLE 1
Excipients used

| | | | Pharmacopoeia |
|---|---|---|---|
| Excipient | Trade name | Function | Reference |
| Lactose monohydrate | Pharmatose ® | Diluent | Ph. Eur.-NF |
| 200 mesh | 200M | | |
| Microcrystalline | Avicel ® PH-101 | Diluent | Ph. Eur.-NF |
| cellulose 50 μm | | | |
| Microcrystalline | Avicel ® PH-102 | Diluent | Ph. Eur.-NF |
| cellulose 90 μm | | | |
| Hypromellose 6 | Methocel ™ E6 | Binder | Ph. Eur.-USP |
| mPa · s | | | |
| Crospovidone type A | Kollidon ® CL | Disintegrant | Ph. Eur.-NF |
| Magnesium stearate | Ligamed MF-2-V | Lubricant | Ph. Eur.-NF |
| vegetable | | | |
| Opadry ® QX | Opadry ® QX | Film-coating | Internal |
| 321A230043 | 321A230043 | agent | Monograph |
| orange^{a} | orange | | |

^{a}Opadry ® orange QX 321A230043 is composed of 40.0% macrogol polyvinyl alcohol grafted copolymer (Ph. Eur.-NF), 27.5% talc (Ph. Eur.-USP), 20.2% titanium dioxide (E171) (Ph. Eur.-USP), 4% glycerol monocaprylocaprate type 1 (Ph. Eur.-NF)/mono/diglycerides (Food Chemical Codex)/glycerol of fatty acids, 3.8% yellow ferric oxide (E172) (NF), 3.5% polyvinyl alcohol partly hydrolyzed (Ph. Eur.-USP) and 1.0% red ferric oxide (E172) (NF).

### Wetting and Granulation Study

In order to achieve comparative wetting and granulation curves by limiting the consumption of active ingredients and in order to allow rapid and efficient implementation, two wetting and granulation methods were tested at small scale, a multiple addition method and variable mix time method, as described below. The two methods were tested with a placebo mixture or a mixture containing Compound 1. The excipients used for this study are described in Table 1.

The centesimal compositions of the placebo and Compound 1 formulations used in this study are described in Tables 2 and 3, respectively.

TABLE 2
Centesimal composition of the placebo formulation

| | |
|---|---|
| Components | Centesimal |
| Placebo formula | composition (%) |

Internal phase

| | |
|---|---|
| Lactose monohydrate | 38.833 |
| Microcrystalline cellulose 50 μm | 34.833 |
| Hypromellose 6 mPa · s | 2.000 |
| Crospovidone Type A | 1.500 |

External phase

| | |
|---|---|
| Microcrystalline cellulose 90 μm | 20.983 |
| Crospovidone Type A | 1.500 |
| Magnesium stearate | 0.350 |
| core tablets | 100.000 |

TABLE 3
Centesimal compositions of Compound 1 formulation

| | Centesimal composition (%) | | |
|---|---|---|---|
| | Formulation name | | |
| | Formula A | Formula B | Formula C |
| | Ratio Lactose/microcrystalline cellulose | | |
| Components | 40/60 | 40/60 | 60/40 |

Internal phase

| | | | |
|---|---|---|---|
| Compound 1 | 10.000 | 15.00 | 15.00 |
| Lactose monohydrate 200 mesh | 28.833 | 23.50 | 35.15 |
| Microcrystalline cellulose 50 μm | 34.833 | 35.15 | 23.5 |
| Hypromellose 6 mPa · s | 2.000 | 2.00 | 2.00 |
| Crospovidone Type A | 1.500 | 1.50 | 1.50 |
| Total internal phase | 77.166 | 77.15 | 77.15 |

External phase

| | | | |
|---|---|---|---|
| Microcrystalline cellulose 90 μm | 20.983 | 21.00 | 21.00 |
| Crospovidone | 1.500 | 1.50 | 1.50 |
| Magnesium stearate | 0.350 | 0.35 | 0.35 |
| talc | — | — | — |
| Final composition | 100.000 | 100.000 | 100.000 |

Wetting and granulation tests were carried out on a scale of about 25 g using a Caleva type granulator. The placebo mixture was at a scale of 1000 g and the Compound 1 mixtures were at a scale of 75 g or 250 g. These mixtures were then divided to perform granulation tests at 25 g and to assess reproducibility with the same initial batch.

The internal phase mixtures were prepared as follows.

Step 1: All components were weighed according to the compositions of Tables 2 and 3.

Step 2: A solution of hypromellose 8% in demineralized water, was prepared the day before under strong stirring, and the solution was diluted to the desired concentration of hypromellose. This step was not performed where hypromellose was added in the dry mixture.

Step 3 for the placebo mixture: The first half of lactose monohydrate was sifted together with microcrystalline cellulose and crospovidone type A. Then the sieve was rinsed with the second half of lactose monohydrate through a sieve with a 0.6 mm mesh opening.

Step 3 for the Compound 1 mixture: Compound 1 was sifted with a first half of lactose monohydrate, then microcrystalline cellulose, hypromellose 6 mPa·s, and crospovidone type A. Then the sieve was rinsed with the second half of lactose monohydrate through a sieve with a 0.6 mm mesh opening.

Step 4 for the placebo mixture: The sieved excipients of Step 3 were mixed for 10 minutes at 7 rpm in a CMA turning mixer equipped with a 5 L tank.

Step 4 for the Compound 1 mixture: The sieved excipients of Step 3 were mixed for 10 minutes at 32 rpm in a Turbula® mixer.

Two wetting and granulation methods were tested, a multiple addition method and variable mix time method, as described below.

Multiple addition method: The Caleva tank was filled with the desired amount of powder (V0=0.412 mass powder=24.7 g) and a 4.42% HPMC solution in demineralized water was prepared. The method used the following parameters:
rotor speed=50 rpm
Mix Time=30 seconds
Log Time=30 seconds
Run number=33
Duration of the test=35 minutes
Addition of 1 mL of HPMC solution every 60 seconds

Total volume added=31 mL

Variable mix time method: The Caleva tank was filled with the desired amount of powder (V0=0.412 mass powder=24.7 g) and wet with HPMC solutions in demineralized water prepared at different concentrations. The method used the following parameters:
rotor speed=50 rpm
Mix Time=40 seconds
Log Time=20 seconds
Run number=12
Stirring time=10 minutes
HPMC floor: 8%, 5.51%, 4.42%, 3.63%, 2.91% and volume was adapted to have 2% HPMC in the final formula.

Granule drying was carried out in an tray dryer at about 50° C. for a batch of about 25 g.

Tests were performed on Compound 1 mixtures by testing several batches of active ingredients and different formulations (Formula A, B and C as described in Table 3) as well as two different modes of introduction of HPMC (in wetting liquid A or in dry mixture B), as described in Table 4A. Table 4B describes the placebo batches. Active Pharmaceutical Ingredient (API) Batch extensometry is described in Table 4C.

TABLE 4A
Formulations with different modes of HPMC introduction

| | Formula A | Formula A | Formula A | Formula B | | Formula C | Formula C | | Formula B |
|---|---|---|---|---|---|---|---|---|---|
| | Batch 1 | Batch 2 | Batch 3 | Batch 2 | | Batch 2 | Batch 1 | | Batch 1 |
| Internal phase | 24.4 g | 24.4 g | 24.4 g | 24.1 g | | 24.1 g | 25 | g | 25 g |
| scale | | | | | | | | | |
| Active | 3 | 4 | 5 | 2 | | 2 | 2 | | 2 |
| Pharmaceutical | | | | | | | | | |
| Ingredient | | | | | | | | | |
| (API) Batch | | | | | | | | | |
| HPMC | A | A | A | B | | B | B | | A |
| in the wetting | | | | | | | | | |
| liquid (A) or | | | | | | | | | |
| in the dry | | | | | | | | | |
| mixture (B) | | | | | | | | | |
| Compression | N/A | N/A | N/A | Stylcam | | N/A | Stylcam | | N/A |
| Format | N/A | N/A | N/A | 7 × 3.8 | | N/A | 7 × 3.8 | | N/A |
| Dose | | | | 10 | mg | | 10 | mg | |
| Mass of tablets | | | | 67 | mg | | 67 | mg | |

TABLE 4B
Description of placebo batches

| | Placebo | Placebo |
|---|---|---|
| | Batch 1 | Batch 2 |
| Internal phase scale | 24.7 g | 24.7 g |
| | (internal phase | (internal phase |
| | mixture 1000 g) | mixture 500 g) |
| HPMC | A | A |
| In the wetting liquid (A) | | |
| or in the dry mixture (B) | | |
| Compression | N/A | N/A |

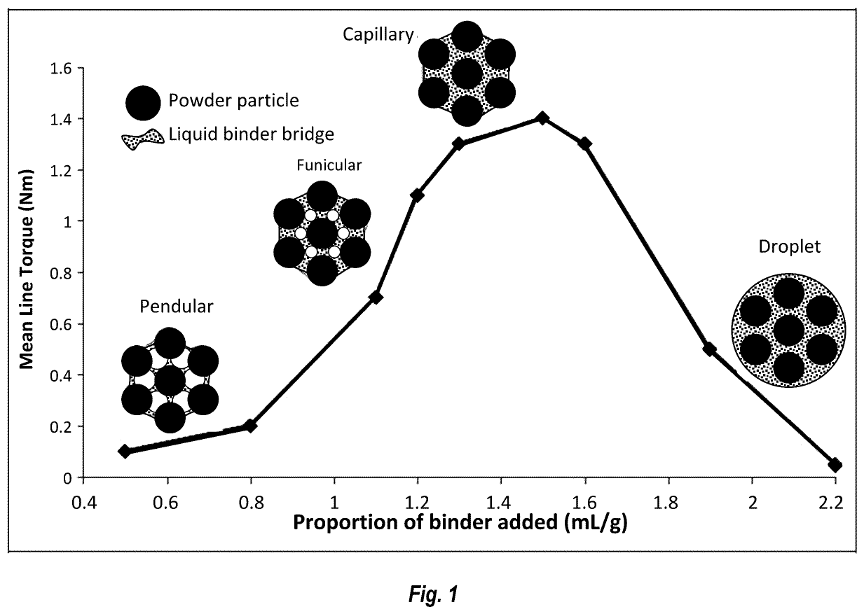

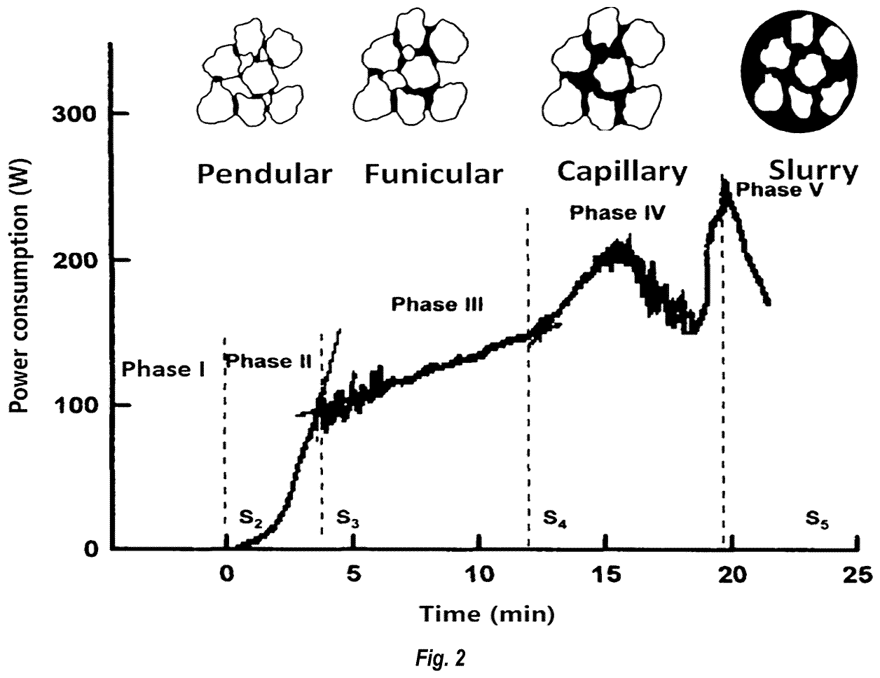

The analysis of the results was performed according to the reference wetting and granulation curves shown in FIG. 1 and FIG. 2. The different states of granulation (pendulum, funicular, capillary and drop) were obtained by plotting the curve of the blade power (torque, N·m) as a function of time or the amount of water added in relation to the weight of the mixture.

250 g or 75 g of internal phase mixtures were prepared for each active ingredient, and tests were carried out at a 25 g scale to evaluate the repeatability (n=3). The concentrations of the wetting HPMC solutions were calculated to obtain (approximately) 2% HPMC in the final formulations.

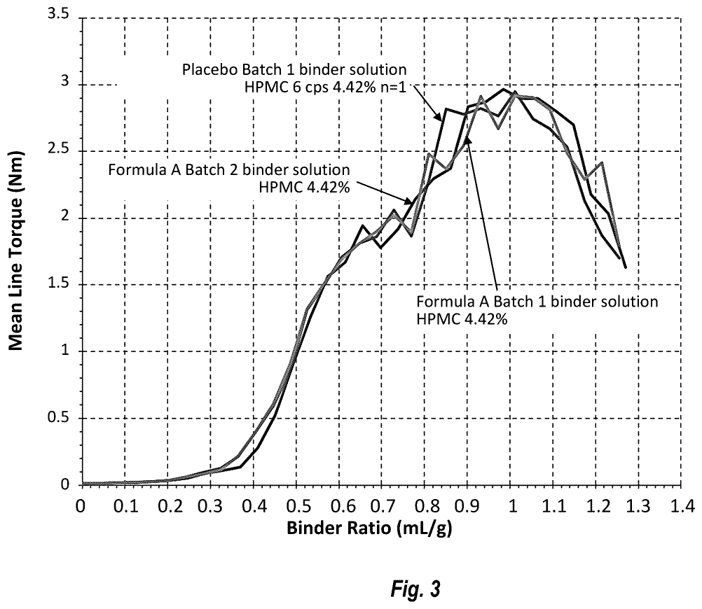

FIG. 3 shows that the behavior of the Formula A Batch 1 and Batch 2 mixtures is equivalent to the placebo with the HPMC in solution in water.

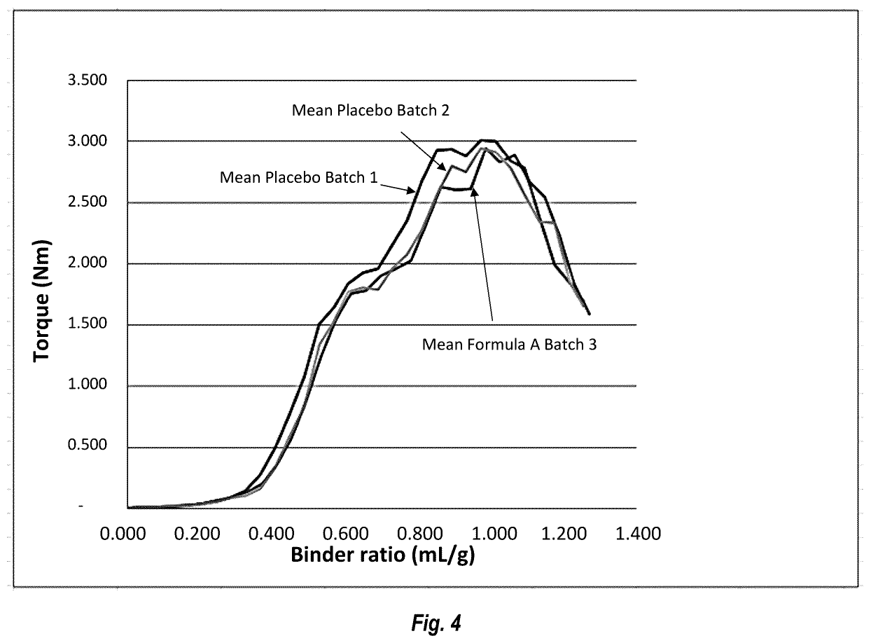

FIG. 4 shows that the behavior of the placebo and Formula A Batch 3 mixtures are equivalent for the granulation step.

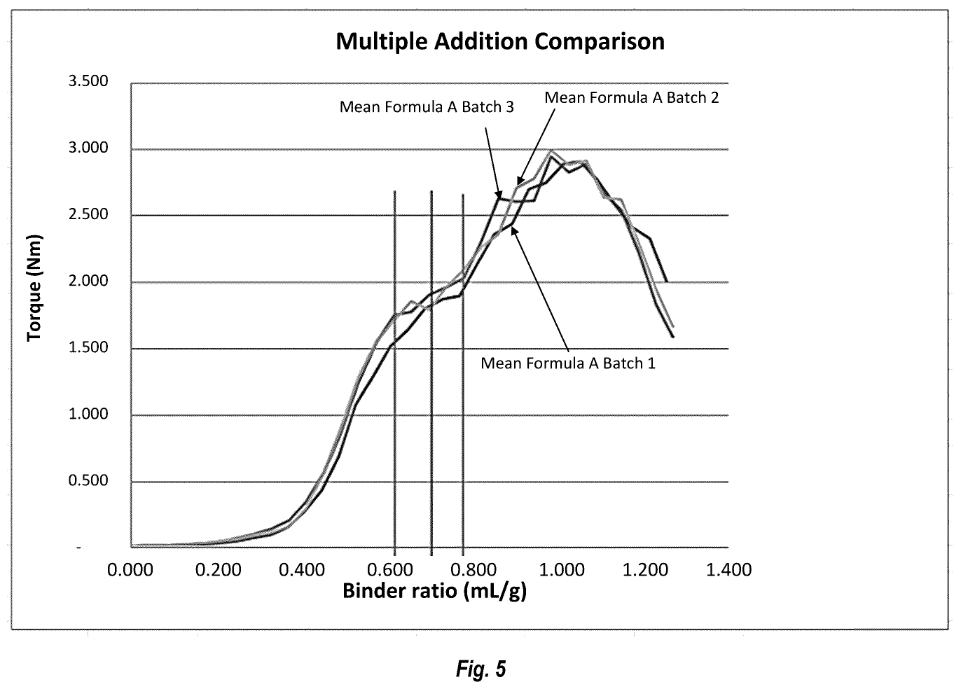

FIG. 5 shows that the behavior of the mixtures of the three batches of Formula A shown in Table 4A are equivalent for the granulation step with where HPMC is in the wetting liquid, and the granulation plateau observed is at the ratio of 0.7 g/mL. The variability of the particle size of the active ingredients, ranging from 10 to 120 μm, for this step does not seem to have an impact. The low densities of the active pharmaceutical ingredient (API) validate the search for a granulation process with the aim of densifying the mixture for compression. As shown in Table 4C, batches of active ingredient used were found to have poor rearrangement, poor cohesion, and little tendency to bond (ejection pressure). The active ingredient from the batches used was rather plastic and very little elastic.

TABLE 4C
API Batches Extensometry 150 MPa

| | Lloyd | | | | | Breaking | Breaking | Ejection |
|---|---|---|---|---|---|---|---|---|
| | bulk | | | | | Work | pressure | pressure |
| API | density | Rearrangement | Elasticity | Relaxation | Plasticity | Hardness | cohesion | sticking |
| Batch | (g/cm³) | flowability | (J/g) | (%) | (%) (Dr) | (J/g) | (Mpa) | (Mpa) |
| 1 | 0.289 | 56 | 2.26 | 13.1 | 1.08 | 9.2 | 1.50 | 11.43 |
| 2 | 0.403 | 36 | 2.19 | 8.3 | 0.81 | 13.4 | 2.24 | 1.61 |
| 3 | 0.290 | 61 | 2.66 | 11.3 | 0.88 | 13.5 | 2.14 | 1.67 |
| 4 | 0.283 | 61 | 2.83 | 11.7 | 0.72 | 10.1 | 2.03 | 1.35 |
| 5 | 0.205 | 68 | 0.70 | 5.37 | 0.63 | 7.8 | 1.51 | 1.70 |

### Comparative Batch Wettability, Drop Angle:

API Batch 5: 66°+/−3º
API Batch 1: 69°+/−3º
API Batch 2: 67°+/−2°
API Batch 3: 90°+/−10°
API Batch 4: 77°+/−3°

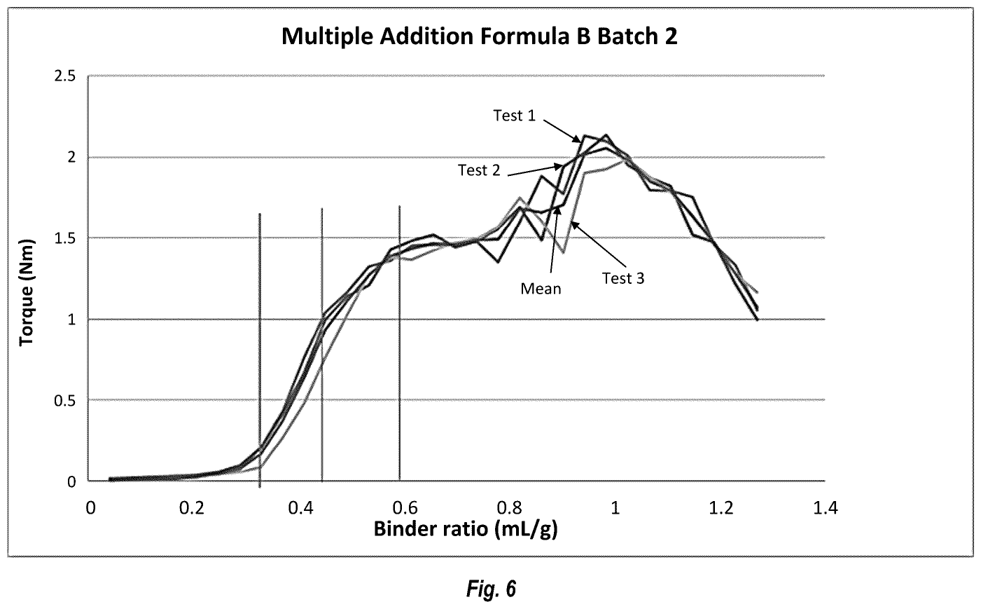

FIG. 6 shows the granulation plateau observed for the Formula B Batch 2 mixture of Table 4A is at the ratio of 0.6 mL/g of added water ratio. It is slightly offset from results shown in FIGS. 3 and 4. This difference is because the % of Compound 1 in Formula B is 15% compared to the batches of Formula A, which contain 10% Compound 1. The batch of active ingredient, API Batch 2, was also different, and these properties can have an impact on the behavior in wetting and granulation.

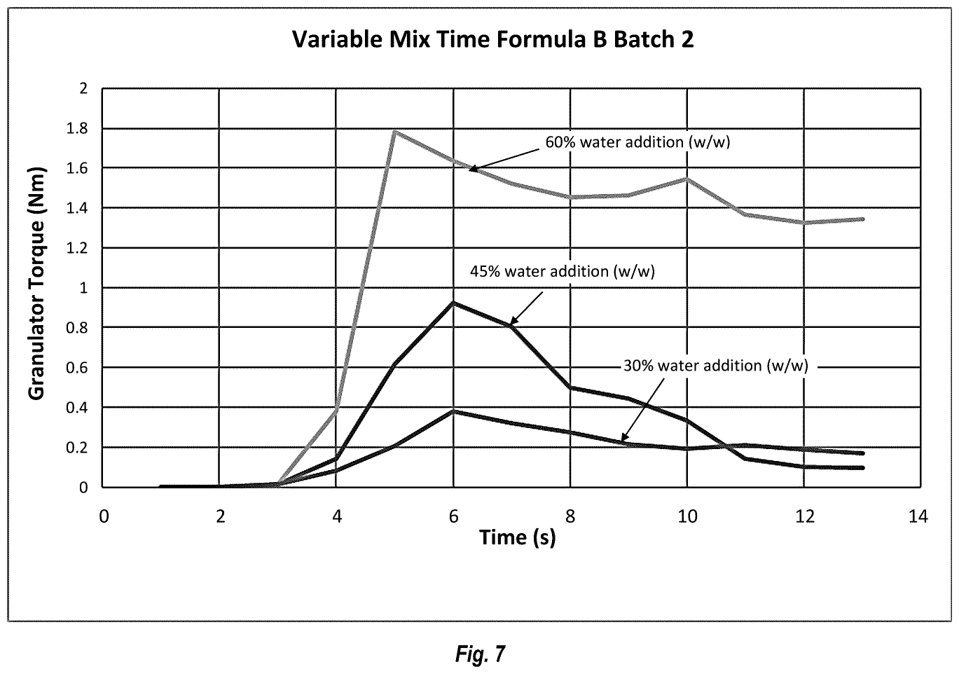

The analysis of the curves in FIG. 7 resulted in the selection of around 45% percent water for anchorage. Formula B Batch 2 contained a lactose/cellulose ratio equal to 40/60, the wetting was carried out with water, and the HPMC was in the dry internal phase mixture.

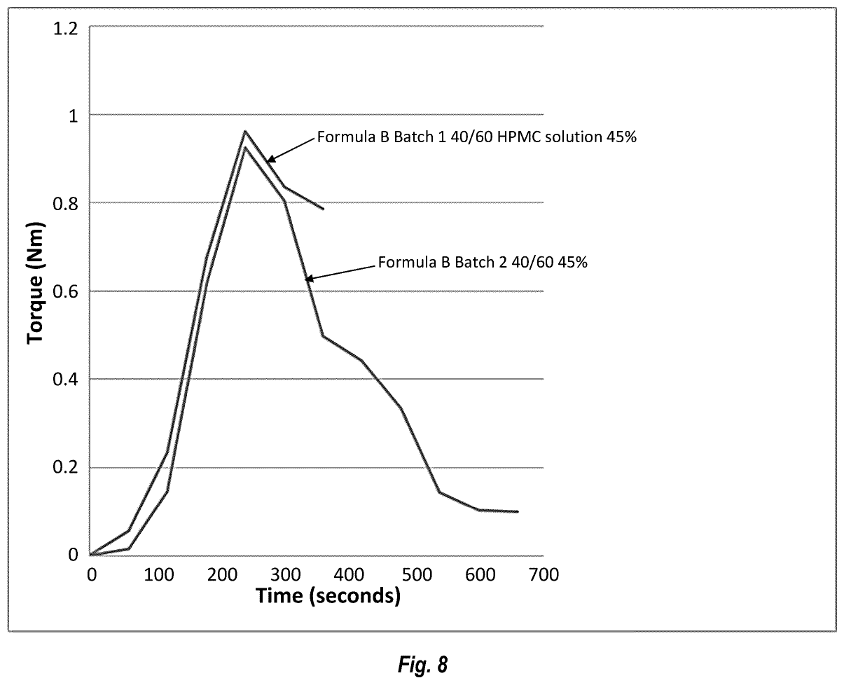

FIG. 8 compares Formula B Batch 1 and Formula B Batch 2, which had the same formulation, including a lactose/cellulose ratio of 40/60, but HPMC was introduced into the wetting solution in Formula B Batch 1 and into the dry mixture for Formula B Batch 2. As the slope of the curves are the same, the addition of HPMC directly in the powder mixture was selected for the purpose of process simplification and industrial practices.

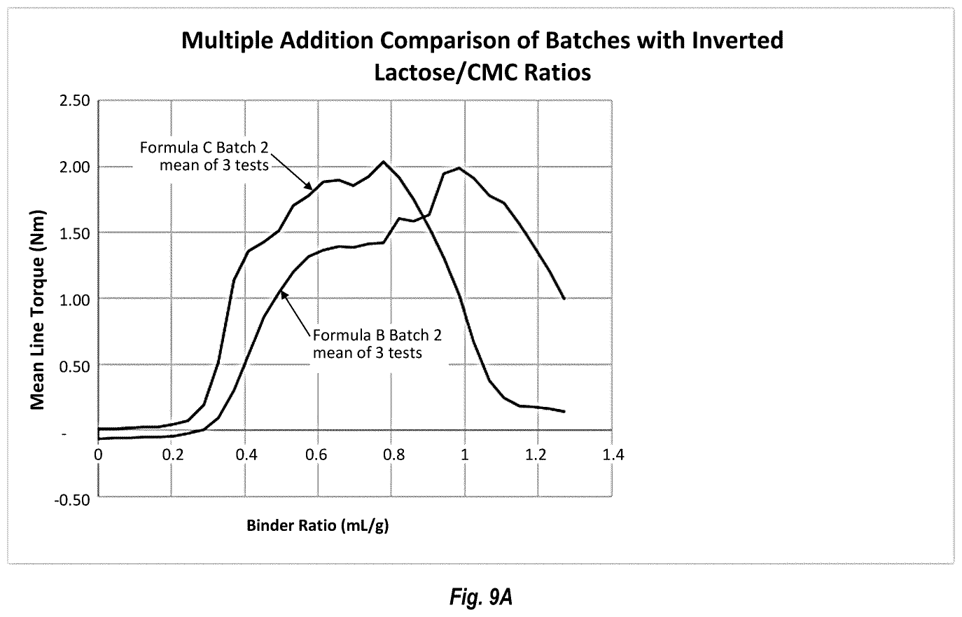

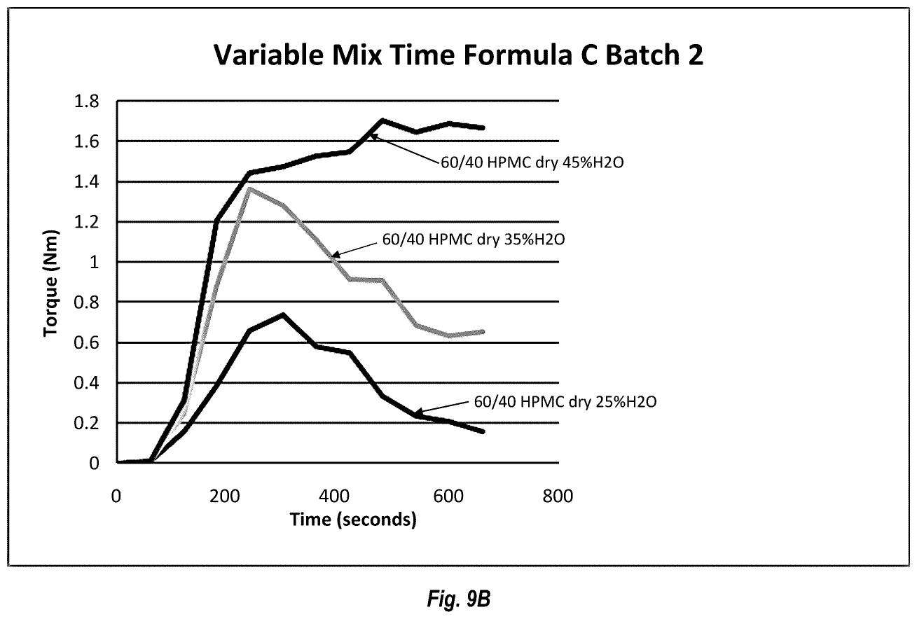

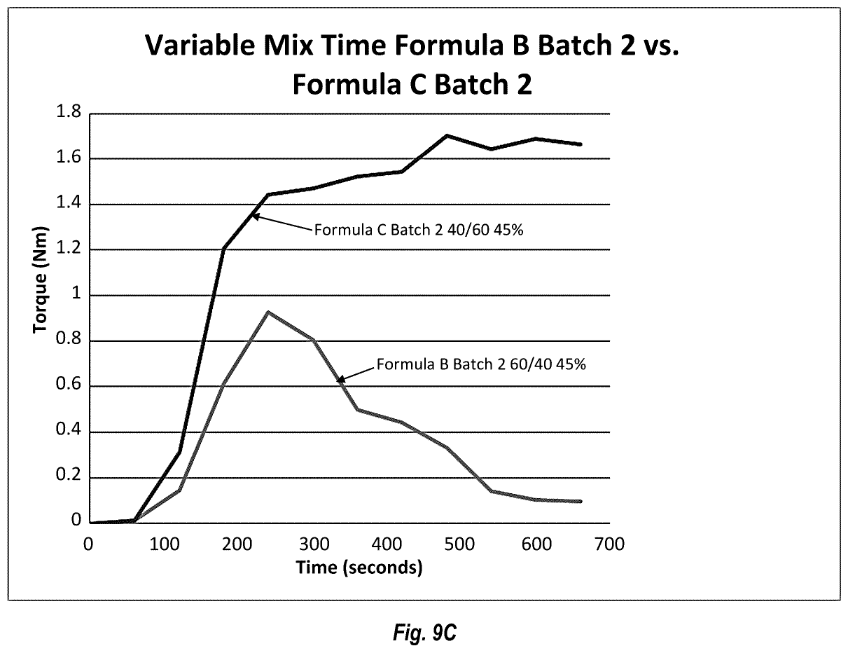

FIGS. 9A, 9B, and 9C compare the curves of Formula B Batch 2 (lactose/cellulose ratio 40/60) and Formula C Batch 2 (lactose/cellulose ratio 60/40). These figures show that the wetting and granulation curves are not similar for different lactose/cellulose ratio formulas. The ideal amount is around 35% for the 60/40 lactose/cellulose ratio while it is around 45% for the 40/60 ratio. Based on these results a 60/40 lactose/cellulose ratio was selected to bring more cohesion and compressibility to the mixture. Another advantage of this ratio is a shorter drying time because less water is required. The difference in behavior was assessed during the compression step.

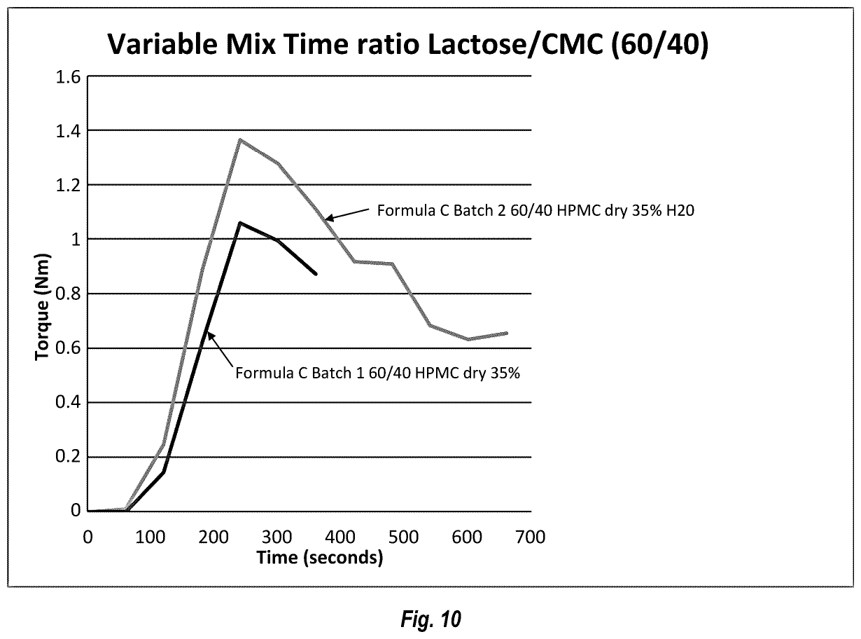

Batch repeatability was assessed by testing Formula C Batches 1 and 2 using the same process and 35% water. The results are shown in FIG. 10.

For Formula B Batch 2 and Formula C Batch 1, the final mixture was manufactured and compression tests were carried out on Stylcam with a simulation of the Korsch XL100, speed 20 rpm, forced feed and punches of size 7×3.8 mm engraved ‘6’. The characteristics of the core tablets are shown in Tables 5 and 6.

TABLE 5
Characteristics of 10 mg core tablets of Formula
B Batch 2, tablets 5, 7, 9 kN on Stylcam

| Characteristics | Target limits | 5 kN | 7 kN | 8 kN |
|---|---|---|---|---|
| Average mass | 67 mg | 67.0 | 66.8 | 65.0 |
| (mg) | Min-max | 66.0-68.7 | 66.0-67.3 | 64.1-66.4 |
| Hardness | For | 89 | 89 | 83 |
| (N) (n = 10) | information | [77-94] | [85-93] | [77-87] |
| Thickness | Mean | 2.44 | 2.41 | 2.37 |
| (mm) (n = 10) | [min-max] | [2.41-2.48] | [2.39-2.41] | [2.33-2.41] |

TABLE 6
Characteristics of 10 mg core tablets of Formula
C Batch 1, tablets 5, 7, 9 kN on Stylcam

| Characteristics | Target limits | 5 kN | 7 kN | 9 kN |
|---|---|---|---|---|
| Average mass | 67 mg | 64.9 | 65.6 | 66.8 |
| (mg) | Min-max | 63.3-67.3 | 64.9-66.2 | 66.5-67.1 |
| Hardness | For | 98 | 105 | 120 |
| (N) (n = 10) | information | [89-104] | [102-111] | [115-127] |
| Thickness | Mean | 2.38 | 2.38 | 2.41 |
| (mm) (n = 10) | [min-max] | [2.34-2.45] | [2.33-2.42] | [2.31-2.46] |

The compression profiles show a drop in hardness at 8-9 kN forces for the Formula B Batch 2 mixture (40/60 ratio) compared to the Formula C Batch 1 mixture.

Based on the granules and final blend properties of the formulations and compression results described in this example, the following selections were used in the preparation of the formulations a lactose/cellulose ratio of 60/40 and use of 35% water during the wetting step of the Compound 1 internal phase mixture containing the HPMC in the dry mixture.

### Example 2: Process for Preparing Film-Coated Tablets Containing Compound 1

Five tablet formulations were developed, as shown in Table 7.

TABLE 7
Comparison of batches of Compound 1 tablets

| Components | Centesimal composition (%) | | | | |
|---|---|---|---|---|---|
| Formulation name | Formula A | Formula B | Formula C | Formula D | Formula E |
| Ratio Lactose/ | 40/60 | 40/60 | 60/40 | 60/40 | 60/40 |
| microcrystalline | | | | | |
| cellulose | | | | | |
| Internal phase | | | | | |
| Compound 1 | 10.000 | 15.00 | 15.00 | 15.00 | 15.00 |
| Lactose monohydrate | 28.833 | 23.50 | 35.15 | 32.15 | 35.00 |
| 200 mesh | | | | | |
| Microcrystalline | 34.833 | 35.15 | 23.5 | 23.5 | 23.50 |
| cellulose 50 μm | | | | | |
| Hypromellose 6 mPa · s | 2.000 | 2.00 | 2.00 | 2.00 | 2.00 |
| Crospovidone Type A | 1.500 | 1.50 | 1.50 | 1.50 | 1.50 |
| Total internal phase | 77.166 | 77.15 | 77.15 | 74.15 | 77.00 |
| External phase | | | | | |
| Microcrystalline | 20.983 | 21.00 | 21.00 | 21.00 | 21.00 |
| cellulose 90 μm | | | | | |
| Crospovidone | 1.500 | 1.50 | 1.50 | 1.50 | 1.50 |
| Magnesium stearate | 0.350 | 0.35 | 0.35 | 0.35 | 0.50 |
| talc | — | — | — | 3.00 | — |
| Final composition | 100.000 | 100.000 | 100.000 | 100.000 | 100.000 |

Several batches with qualitatively and quantitatively different formulations were manufactured at a 300 g scale in a 1 L granulation tank in order to compare the properties of mixtures and tablets. These are Formula E Batch 1, Formula C Batch 3, Formula D Batch 1 and Formula E Batch 2. The excipients used for this study are described in Table 1.

Step 1: All components were weighed according to the tablet formulations of Table 1.

Step 2: Compound 1 was sifted with a first half of lactose monohydrate, then microcrystalline cellulose, hypromellose 6 mPa·s, and crospovidone type A, then the second half of lactose monohydrate through a sieve with a 0.6 mm opening.

Step 3: The sieved ingredients were loaded into the granulator tank at high shear speed and mix for 5 min to 250 r/min for the tank bottom blade and 0 r/min for the chopper.

Step 4: The ingredients were wet at a flow rate of about 80 mL/min to about 100 mL/min (fast flow) by mixing with about 35% (w/w) of water at 250 r/min for the tank bottom blade and 0 r/min for the chopper, and granulated for about 2 minutes with a speed of 1500 r/min for the chopper.

Step 5: The granules obtained were dried in a fluidized air bed at an inlet temperature of 60° C. and an air flow of about 40 m3/h until the water content was close to the amount before granulation, for example, ranging from about 23 minutes to about 35 minutes to obtain a loss on drying (LOD) equivalent to the initial dry mixture ranging from about 1.48% to about 1.94%, or ranging from about 1.5% to about 1.9%.

Step 6: The resulting granules were calibrated on a 0.991 mm opening grid using a minimum rotor speed of 750 rpm.

Step 7: The external phase excipients (microcrystalline cellulose 90 μm and crospovidone) were sieved on a sieve having a 0.6 mm opening.

Step 8: The external phase excipients were added to the calibrated granules in step 6 and mix in a blender for 20 minutes at 10 r/min.

Step 9: The magnesium stearate was sieved on a sieve with an opening of 0.6 mm.

Step 10: The sieved magnesium stearate was added to the calibrated granules and mixed for 10 minutes at 6 r/min to obtain a homogeneous mixture.

Step 11: The lubricated granules were compressed on Korsch XL 100 press equipped with 4 punches, 3.8×7 mm for 10 mg dosage strength tablets and 12.65×5.75 mm for 60 mg dosage strength tablets, using a feeder speed of 10 rpm and a turret speed of 20 rpm.

Step 12: A 30% Opadry QX film coating suspension was prepared.

Step 13: For Formula E Batches 1 and 2, the 10 mg dosage strength and 60 mg dosage strength tablets were film coated to achieve a mass gain of 3% using the following parameters: a 1.2 mm nozzle, an inlet temperature of 60° C., an airflow of 50 m³/h, a drum speed of 2 rpm in preheating with the speed adjusted according to the bed of the tablets, a suspension flow rate of around <5 g/min, a spray pressure of 1.3 bar, and an angle pressure of 1.3 bar.

Table 8 shows the manufacturing parameters used during batch manufacturing.

TABLE 8
Parameters used during manufacture of tablets containing Compound 1

| Step | Manufacturing | Tests and controls | Formula E | Formula C | Formula D | Formula E | | | |
|---|---|---|---|---|---|---|---|---|---|
| No. | step | in progress | Batch 1 | Batch 3 | Batch 1 | Batch 2 | | | |
| 2 - | Sieving | Grid size (mm) | 0.6 | 0.6 | 0.6 | 0.6 | | | |
| 3 - | Mixture | Speed (r/min) | 250 | 250 | 250 | 250 | | | |
| | | duration(min) | 5 | 5 | 5 | 5 | | | |
| | | Residual moisture (%) | 2.56 | 2.56 | 2.89 | Nr | | | |
| 4 - | Wet granulation | Speed (r/min) | 250 | 250 | 250 | 250 | | | |
| | | duration(min) | 2 | 2 | 2 | 2 | | | |
| | | Quantity of water (%) | 35 | 35 | 35 | 35 | | | |
| | | Water flow rate (ml/min) | 83 | 86 | 83 | 82 | | | |
| 5 - | Drying | Inlet temperature (° C.) | 60 | 60 | 60 | 60 | | | |
| | | Outlet temperature (° C.), | 38 | 40.5 | 37.7 | 36.3 | | | |
| | | at the end of drying | | | | | | | |
| | | Airflow (m3/h) | 40 | 40 | 40 | 40 | | | |
| | | Duration(min) | 35 | 35 | 25 | 23 | | | |
| | | Residual moisture (%) | 1.94 | 1.87 | 1.69 | 1.48 | | | |
| 6 - | Calibration | Mesh aperture (mm) | 0.991 | 0.991 | 0.991 | 0.991 | | | |
| 7 - | Sieving | Grid size (mm) | 0.6 | 0.6 | 0.6 | 0.6 | | | |
| 8 - | Final blend | Speed (r/min) | 20 | 20 | 20 | 20 | | | |
| | | Duration (min | 10 | 10 | 10 | 10 | | | |
| 9 | Sieving | Grid size (mm) | 0.6 | 0.6 | 0.6 | 0.6 | | | |
| 10 - | Final blend | Speed (r/min) | 6 | 6 | 6 | 6 | | | |
| | | Duration(min) | 10 | 10 | 10 | 10 | | | |
| 11 - | Compression | Pre-compression force (kN) | ≤1 | ≤1 | ≤1 | ≤1 | | | |
| | | Compressive force (kN) | 5 to 9 kN | 5 to 8 kN | 5 to 9 kN | 7 kN | | | |
| | | | for 7 × 3.8 | for 7 × | for 7 × | for 7 × | | | |
| | | | mm and | 3.8 mm | 3.8 mm | 3.8 mm | | | |
| | | | 12.65×5.75 | 7 to 14 kN | 5 to 9 kN | 12 kN | | | |
| | | | mm | for 12.65 × | for 12.65 × | for 12.65 × | | | |
| | | | | 5.75 format | 5.75 mm | 5.75 mm | | | |
| | | | | | format | format | | | |
| | | Feeder speed (r/min) | 10 | 10 | 10 | 10 | | | |
| | | Turret speed (r/min) | 20 | 20 | 20 | 20 | | | |
| 13 - | Film-coating | | | | | 10 | mg | 60 | mg |
| Dosage | | | | | | 136 | g | 97 | g |

| | | | | |
|---|---|---|---|---|
| batch size | | | | |
| 13 - | Film-coating | Nozzle diameter | 1.2 | 1.2 |
| | | Spray and angle pressure (bars) | 1.3, 1.3 | 1.3, 1.3 |

| | | | |
|---|---|---|---|
| Suspension flow rate (g/min) | 5 | 2.4-4.6 | g/min |

| | | |
|---|---|---|
| Drum speed (rpm) | 12-14 | 10 |
| Inlet temperature (° C.) | 60 | 60 |
| Outlet temperature (° C.) | 42-45 | 46-49 |
| Inlet air flow (m3/h) | 50 | 50 |
| Tablet Bed Temperature (° C.) | 35-39 | 40-43 |
| Mass gain (%) | >3% | >3% |
| Duration (min) | 10 min | 15 min |
| Visual inspection | Homogeneous, | Homogeneous, |
| | smooth, crisp | smooth, crisp |
| | engraving | engraving |

^{a} Only Formula E Batch 2 was film-coated.

### Tests and Evaluation Procedures

The following tests were performed on the final mixture prepared in Step 10:
Residual moisture by loss on desiccation (Ph. Eur. 2.2.32) carried out on 1 g of mixture with a thermobalance at 95 º C for 20 minutes.
Homogeneity of mixture carried out on 3 samples: high, middle, low.
Bulk density (m/V in g/mL) (Ph. Eur. 2.9.34) determined from the volume of 100 g of powder obtained in a graduated specimen of 250 mL before settlement and after 10, 500 and 1250 settlements.
Compressibility index (or Carr index) (%) is calculated according to the following formula: 100×[(V₀−V_{f})/V₀], V₀ being the apparent unpacked volume and V_{f}, the final volume causing the powder to settle until a constant volume is obtained. The Hausner index is calculated according to the following formula: V₀/V_{f}. The generally accepted scale of flowability is presented in Table 9 according to Ph. Eur. 2.9.36.
Flow (Ph. Eur. 2.9.16) made on 100 g of powder.
Flow index through an orifice (mm) (Ph. Eur. 2.9.36) determined on a Flodex device on 50 g of powder from the smallest orifice diameter through which the sample can flow over 3 successive tests.
Particle size distribution by sieving (Ph. Eur. 2.9.38) carried out on 25 g of powder with sieves of opening size of 850, 425, 250, 180, 150, 106.75, <75 μm.

The results are expressed as a percentage of powder (w/w) not retained on the sieve.

TABLE 9
Scale of flowability

| Compressibility index (%) | Flowability | Hausner Index |
|---|---|---|
| 1-10 | Excellent | 1.00-1.11 |
| 11-15 | Good | 1.12-1.18 |
| 16-20 | Pretty good | 1.19-1.25 |
| 21-25 | Passable | 1.26-1.34 |
| 26-31 | Mediocre | 1.35-1.45 |
| 32-37 | Very poor | 1.46-1.59 |
| >38 | Extremely poor | >1.60 |

The following tests were performed on the core tablets prepared in Step 11:
Average mass made on 10 tablets being compressed
Mass uniformity (Ph. Eur. 2.9.5) performed on 20 tablets during compression
Hardness (Ph. Eur. 2.9.8) achieved on 10 compressed oriented lengthwise during compression
Thickness measured on 10 tablets during compression course
Friability test (Ph. Eur. 2.9.7) carried out on not less than 6.5 g of tablets after 4 and 30 minutes at 25 r/min at the beginning, middle and end of compression
Disaggregation (Ph. Eur. 2.9.1) carried out on 10 tablets at the beginning, middle and end of compression
Dissolution test carried out on 6 tablets according to the method described in this example.

The following tests were performed on the film-coated tablets prepared in Step 13:
Average mass made on 10 tablets being compressed
Mass uniformity (Ph. Eur. 2.9.5) performed on 20 tablets during compression
Hardness (Ph. Eur. 2.9.8) performed on 10 length-oriented tablets during compression
Thickness measured on 10 tablets being compressed
Friability test (Ph. Eur. 2.9.7) performed on not less than 6.5 g of tablets after 4 and 30 minutes at 25 r/min at the beginning, middle and end of compression
Disaggregation (Ph. Eur. 2.9.1) carried out on 10 tablets at the beginning, middle and end of compression
Dissolution test carried out on 6 tablets according to the method described in this example.

The following evaluation criteria were applied:
Process monitoring according to defined parameters
Granulation state: funicular/capillary
Properties of calibrated grains: densities, particle size
Properties of the final mixture: flow, densities, particle size
Analysis of Carr index values and Hausner ratios
Good compressibility of the final mixture
On the film-coating stage, in terms of aesthetics, the controls are as follows: a homogeneous color, a smooth surface, a sharp engraving
Characteristics of core tablets and film-coated tablets: uniformity of mass, thickness, hardness, friability and disintegration time.

Analytical results on film-coated tablets containing Compound 1: uniformity of content and dissolution profiles in accordance with an immediate release type profile.

### Method of Dissolution

The operating conditions for the dissolution method are as follows:

Dissolution: 6 tablets in stage I+6 tablets for stage II

Dissolution analyses according to USP <711> and Ph. Eur. 2.9.3
a. Devices:
   i. Type 2 pallet apparatus
   ii. Visible UV spectrophotometer
b. Conditions of dissolution:
   Rotational speed: 75 rpm for 30 minutes then 150 rpm from 30 minutes (completed) to 45 minutes
   Medium: solution of citric acid and tri-sodium citrate at pH=3.5
   Volume: 900 mL
   Bath temperature: 37° ° C.
   Sampling: every 5 minutes up to 45 minutes
   Wavelogger: 300 nm
   Number of units tested: minimum of 6 units (“Stage I”) to 24 (“Stage III”) if necessary

### Results

TABLE 10
Comparison of batches containing Compound 1

| | Centesimal composition (%) | | | |
|---|---|---|---|---|
| | Formulation names | | | |
| | Formula C | Formula D | Formula E | Formula E |
| | Batch 3 | Batch 1 | Batch 1 | Batch 2^{a} |
| | Ratio Lactose/microcrystalline cellulose | | | |
| Components | 60/40 | 60/40 | 60/40 | 60/40 |
| Active Batch # | 2 | 2 | 2 | 4 |
| % Magnesium Stearate | 0.35 | 0.35 | 0.5 | 0.5 |
| % talc | 0 | 3 | 0 | 0 |
| Internal phase scale | 300 g | 288 g | 299 g | 299 g |

^{a}Tablets dosed at 10 mg (batch size 136 g) and 60 mg (batch size: 97 g) were film-coated with the orange film-coating agent QX

Based on Formulation C loaded to 15% Compound 1 and with the optimized ratio of lactose/cellulose of 60/40, a study was performed to determine the impact of a change in the percent of magnesium stearate or the addition of talc to the formulation on the properties of the granulation mixture and tablets. The addition of colloidal silica was also considered for the purpose of improving flow, but no studies with colloidal silica were performed.

The process applied is simplified and optimized in a preliminary way:
The flow rate of the wetting suspension (water) is fast of the order of 100 ml/min
The % of water is 35%
The speed of the chopper is fast 1500 rpm
The dry grain is directly calibrated in full with the calibrator equipped with a 0.991 mm grid

For these batches, the final granules and mixtures were either partially characterized, or not properly recorded or characterized. For some batches, a bad sampling was performed, so the data are not described because they are not interpretable.

For comparison, only characterizations of 10 mg and 60 mg tablets, manufactured at the force of 7 kN or 12 kN are presented in this example.

The granulation step was carried out with 35% water according to the process of Example 2 and the process parameters described in Table 8.

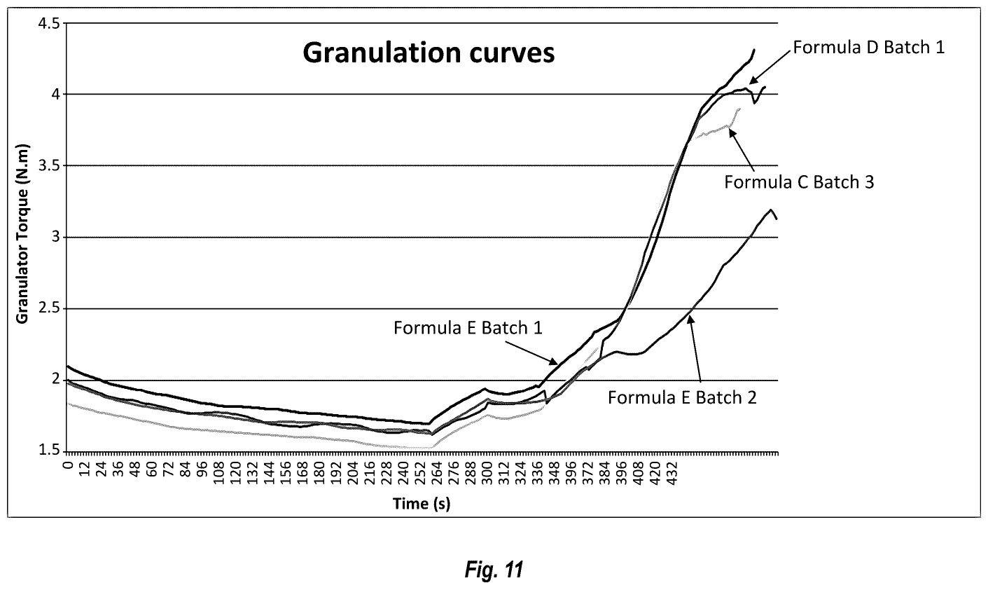

As shown in FIG. 11, the wetting curves Formulation E Batch 1, Formulation C Batch 3, and Formulation D Batch 1 overlap. The curve is slightly different for Formulation E Batch 2. The power is lower after 2 minutes of granulation, and the granulation cost is slower which may be preferable to master this step when scaling up. The difference analyzed is related to the batch of active ingredient. The impact of the physical quality of the active ingredient was studied in the process optimization study.

As shown in Table 11, the drying time varies between 23 and 35 minutes in order to obtain a LOD equivalent to the initial dry mixture, between 1.48 and 1.94%. Drying conditions are similar considering the temperatures and humidity data.

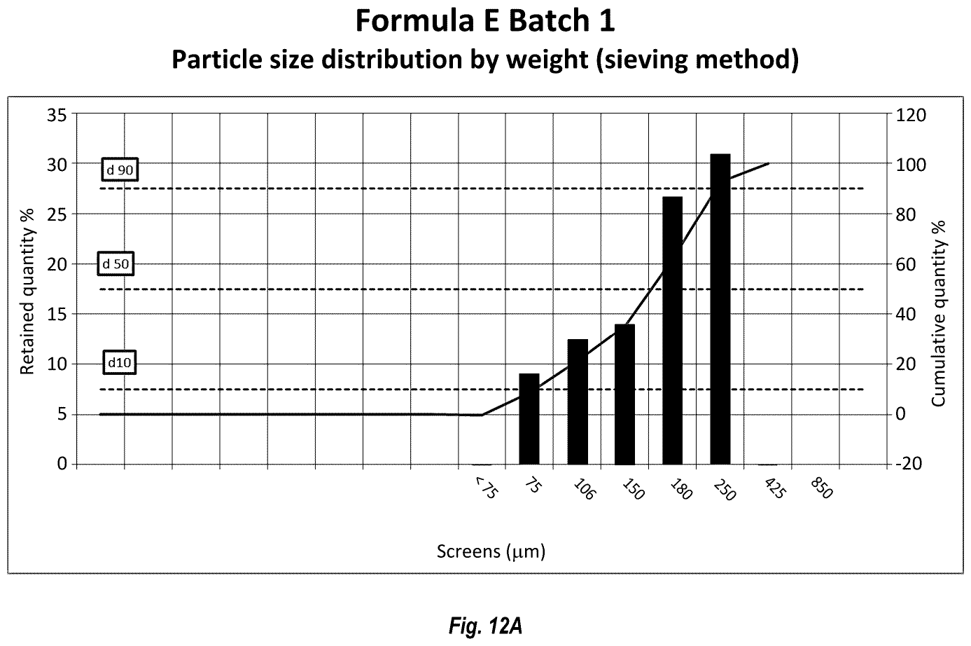

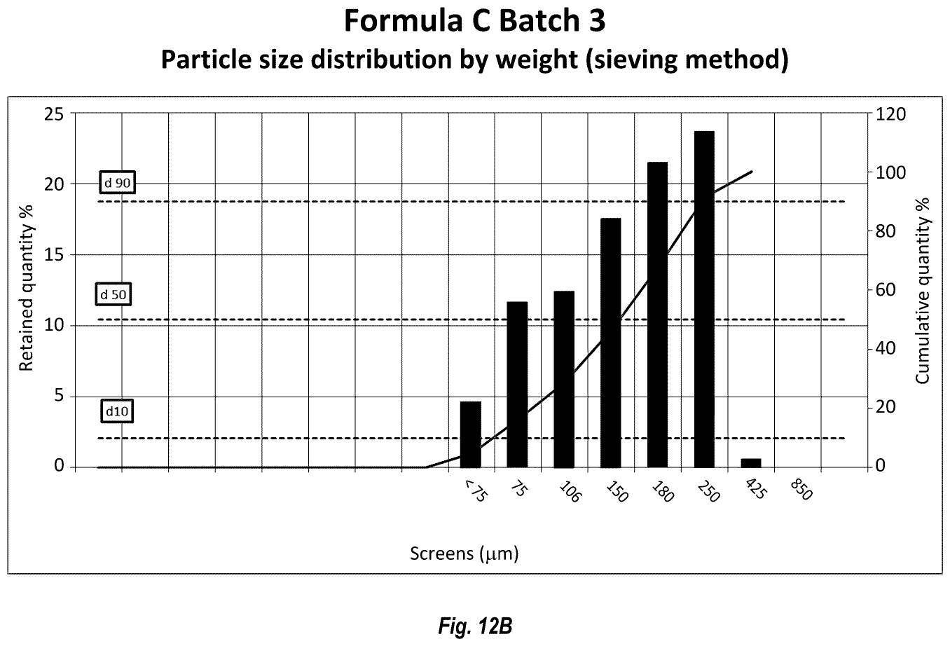

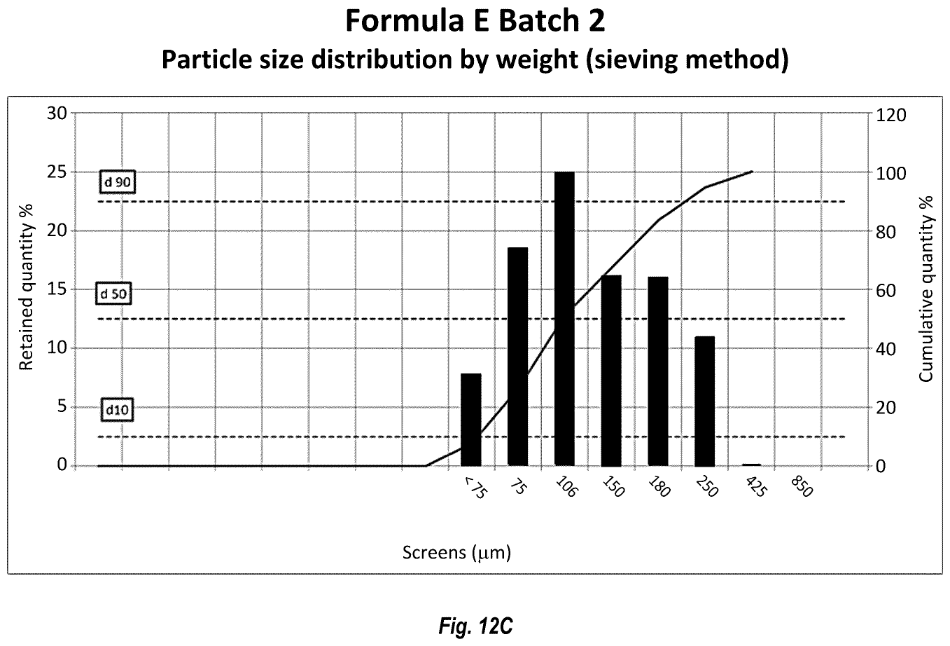

The description of the characteristics of the final mixtures is described Table 12 and FIGS. 12A, 12, and 12C.

TABLE 11
Drying parameters for batches containing Compound 1

| | Batch | | | T ° air | T ° air | HR | HR | DP | DP | LOD (%) |
|---|---|---|---|---|---|---|---|---|---|---|
| | of | Duration | Product | inlet | outlet | input | output | filter | product | Initial |
| Batch | API | (min) | (° C.) | (° C.) | (° C.) | (g/kg) | (g/kg) | (Pa) | (Pa) | Final |
| Formula E | 2 | 35 | 50.0 | 60 | 38.6 | 11.7 | 13.0 | Nr | Nr | 2.56 |
| Batch 1 | | | | | | | | | | 1.94 |
| Formula C | 2 | 35 | 51 | 60 | 40.5 | 8 | 8.9 | 22 | 915 | 2.56 |
| Batch 3 | | | | | | | | | | 1.87 |
| Formula D | 2 | 25 | 49.4 | 60 | 37.7 | 12 | 13.2 | 0 | 932 | 2.89 |
| Batch 1 | | | | | | | | | | 1.69 |
| Formula E | 4 | 23 | 48.3 | 60 | 36 | 8.7 | 9.7 | 11 | 714 | Not |
| Batch 2 | | | | | | | | | | 1.48 |

TABLE 12
Characteristics of the final mixture of batches containing Compound 1

| Batch | Flowability (Copley) | Angle of rest (° C.) | V 10-V500 | Carr Index (%), V10 | Hausner Ratio, V10 |
|---|---|---|---|---|---|
| Formula E | 18 sec (fair) | 32.4 (good) | 10 (good) | 12 (good) | 1.14 (excellent) |
| Batch 1 | funnel:6 sec (good) | | | | |
| Formula C | 20 | 32.6 (good) | 21 (good) | 25 (fair) | 1.33 (fair) |
| Batch 3 | | | | | |
| Formula D | | | NR⁰ | | |
| Batch 1 | | | | | |
| Formula E | 24 (bad) | 32.7 (good) | 20 (good) | 22 (fair) | 1.28 (fair) |
| Batch 2 | | | | | |

^{a} NR: not made or not registered

Table 13 shows the batches characterized have similar values of D10, D50, and D90.

TABLE 13
Comparative particle size by sieving analysis
of batches containing Compound 1

| Batch | D10, μm | D50, μm | D90, μm | Span |
|---|---|---|---|---|
| Formula E | 78 | 167 | 244 | 0.99 |
| Batch 1 | | | | |
| Formula C | 37 | 155 | 245 | 1.34 |
| Batch 3 | | | | |
| Formula E | 48 | 159 | 261 | 1.35 |
| Batch 2 | | | | |

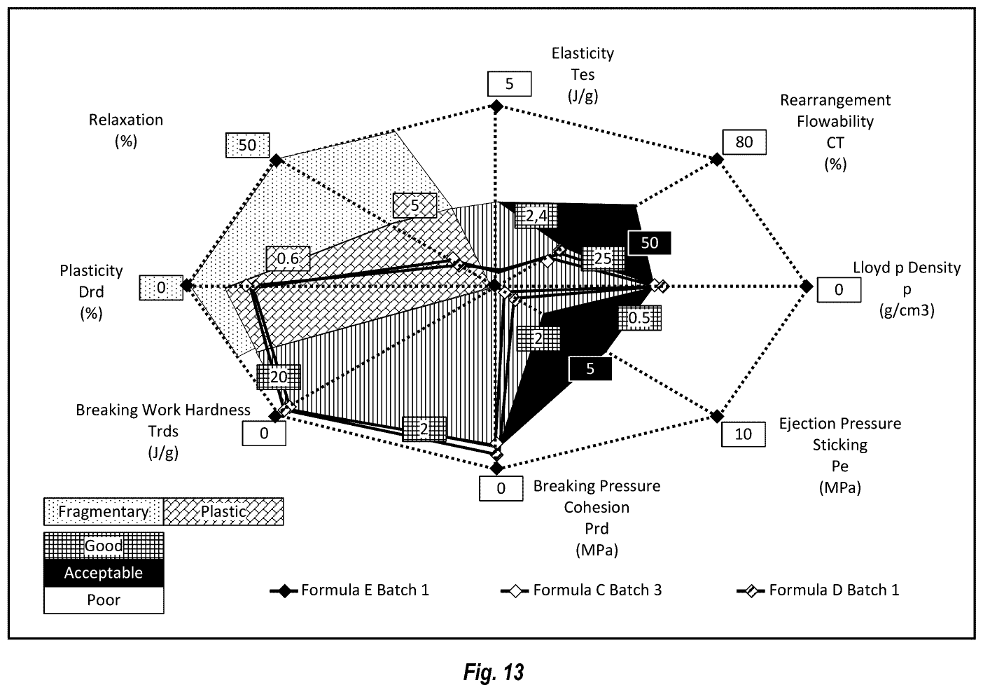

The extensometry profile of the final mixtures of batches Formulation E Batch 1, Formulation C Batch 3, Formulation D Batch 1, and Formulation E Batch 2 was carried out. The results are shown in FIG. 13 and Table 14.

Synthetic analysis of extensometry profiles at 150 MPa show the batches have the following characteristics:
Correct density (around 0.47 g/cm3)/Good rearrangement (<25%) resulting in easy flow for the 3 formulations.
Rather plastic compound (Dr>0.6) at break
Acceptable elasticity since it is below the limit of 2.4 J/g,
Good powder cohesion for Formula E Batch 1/Formula C Batch 3 (Prd of 2.44 and 2.46 MPa>2 MPa) and somewhat inferior powder cohesion for Formula D Batch 1 (Prd of 1.71 Mpa<2 Mpa)
Good ejection because <2 MPa: no major risk of bonding is to be anticipated

In summary, based on the extensometric properties at 150 MPa, the 2 formulations Formulation E Batch 1 and Formulation C Batch 3 are similar, and Formulation D Batch 1, containing talc is somewhat inferior because of a significant loss of cohesion.

TABLE 14
Compression, break, and ejection parameters of batches containing Compound 1

| | | | | | Break | | | Ejection |
|---|---|---|---|---|---|---|---|---|
| | Compression | | | | | Breaking | Breaking | Ejection Pressure |
| | Lloyd ρ | Rearrangement | Elasticity | | Plasticity | Work | Pressure | Sticking |
| | Density | Flowability | Tes | Relaxation | Drd | Hardness | Cohesion | Pe |
| Batch | ρ | C_{T} | (J/g) | (%) | (%) | Trds | Prd | (MPa) |
| Formula E | 0.4707 | 18.88 | 0.4275 | 8.6981 | 1.11 | 21.0801 | 2.44 | 0.37 |
| Batch 1 | | | | | | | | |
| Formula C | 0.4847 | 17.70 | 0.4317 | 10.3423 | 1.01 | 19.6969 | 2.46 | 0.38 |
| Batch 3 | | | | | | | | |
| Formula D | 0.4720 | 22.93 | 0.4823 | 8.5537 | 1.01 | 14.5523 | 1.71 | 0.70 |
| Batch 1 | | | | | | | | |

TABLE 15
Characteristics of 10 mg core tablets, 7 kN tablets

| | | Formula E | Formula C | Formula D | Formula E |
|---|---|---|---|---|---|
| Characteristics | Target limits | Batch 1 | Batch 3 | Batch 1 | Batch 2 |
| Average mass (mg) | 67 mg ± 5 mg | 67.6 ± 1.4 | 65.9 ± 1.6 | 68.8 ± 2.0 | 68.0 ± 2.1 |
| Mass uniformity (n = 20) | Mean (%) | 99.8 | 97.2 | 102.3 | Not scanned |
| | CV (%) | 4.6 | 2.4 | 0.8 | |
| | Goes | 11.1 | 6.9 | Not filled in | |
| Hardness (N) (n = 10) | For information | 90 ± 8 | 83 ± 7 | 73 ± 4 | 72 ± 12.0 |
| Thickness (mm) (n = 10) | Mean [min-max] | 2.43 [2.42-2.44] | 2.37 [234-2.39] | 2.47 [2.45-2.49] | 2.46 [2.42-2.52] |
| Friability after 4 | ≤1 | N/A | 0.31 | N/A | N/A |
| minutes (%) | | | | | |
| Friability after 30 | ≤1 | | 0.77 | | |
| minutes (%) | | | | | |
| Disaggregation (max | <15 | 3 min 27 | 2 min 03 | 1 min 35 | 2 min 24 |
| duration, min) | | | | | |

TABLE 16
Characteristics of 60 mg core tablets, 7 kN tablets

| | | Formula E | Formula C | Formula D | Formula E |
|---|---|---|---|---|---|
| | | Batch 1 | Batch 3 | Batch 1 | Batch 2 |
| Characteristics | Target limits | 7 N | 7 kN | 7 kN | 12 kN |
| Average mass (mg) | 400 mg ± 20 mg | | | | |
| | Mean [min-max] | 404.3 | 404.4 | 406.3 | 392 |
| | | [399.9-406.9] | [401.4-406.8] | [401.5-409.4] | [384-399] |
| Mass uniformity (n = 20) | Mean (%) | 100.9 | 98.7 | 101.1 | 97.25 |
| | CV (%) | 0.5 | 1.6 | 0.6 | 0.85 |
| | Goes | | 3.7 | | |
| Hardness (N) (n = 10) | For information | 184 | 220 | 194 | 261 |
| | | [175-193] | [210-232] | [181-203] | [252-277] |
| Thickness (mm) (n = 10) | Mean [min-max] | 6.16 | 6.10 | 6.19 | 5.67 |
| | | [6.18-6.22] | [6.08-6.11] | [6.15-6.25] | [5.59-5.81] |
| Friability after 4 | ≤1 | 0.09 | 0.23 | 0 | NR⁰ |
| minutes (%) | | | | | |
| Friability almost 30 | ≤1 | 0.24 | 0.68 | 0 | |
| minutes away (%) | | | | | |
| Disintegration (max | <15 | 0 min 31 | 1 min 06 | 1 min 37 | 2 min 10 |
| duration, min) | | | | | |

^{a} NR: not realized

The 10 mg and 60 mg tablets have the desired characteristics. At the end of the study, based on results of the granulation step, mixing, and compressed products, the lactose/cellulose ratio of 60/40 and 0.5% magnesium stearate were selected taking into account scale-up and processability. This formulation corresponds to Formula E Batches 1 and 2.

Tables 17A-17C show the composition of the tablets prepared using this method.

TABLE 17A
Composition of 10 mg and 60 mg dosage strength
tablets containing Compound 1 (Formula E)

| | | | |
|---|---|---|---|
| | Centesimal | Unit | Unit |
| | composition | composition | composition |
| Components | (%) | 10 mg | 60 mg |

Intragranular

| | | | |
|---|---|---|---|
| Tolebrutinib (SAR442168) | 15.00 | 10.00 | 60.00 |
| Lactose monohydrate | 35.00 | 23.45 | 140.00 |
| 200 mesh | | | |
| Microcrystalline cellulose | 23.50 | 15.75 | 94.00 |
| 50 μm | | | |
| Hypromellose 6 mPa · s | 2.00 | 1.00 | 6.00 |
| Crospovidone Type A | 1.50 | 1.35 | 8.00 |
| Total intragranular | 77.00 | | |

Extragranular

| | | | |
|---|---|---|---|
| Microcrystalline cellulose | 21.00 | 14.10 | 84.00 |
| 90 μm | | | |
| Crospovidone | 1.50 | 1.00 | 6.00 |
| Magnesium stearate | 0.50 | 0.35 | 2.00 |
| Composition core tablets | 100.000 | 67 mg | 400 mg |

Filming

| | | | |
|---|---|---|---|
| Opadry ® QX 321A230043 | 3.00 | 2.00 | 12.00 |
| Orange^{a} | | | |
| Film-coated tablet | | 69 mg | 412 mg |
| composition | | | |

^{a}Opadry ® orange QX 321A230043 is composed of 40.0% macrogol polyvinyl alcohol grafted copolymer (Ph. Eur.-NF), 27.5% talc (Ph. Eur.-USP), 20.2% titanium dioxide (E171) (Ph. Eur.-USP), 4% glycerol monocaprylocaprate type 1 (Ph. Eur.-NF)/mono/diglycerides (Food Chemical Codex)/glycerol of fatty acids, 3.8% yellow ferric oxide (E172) (NF), 3.5% polyvinyl alcohol partly hydrolyzed (Ph. Eur.-USP) and 1.0% red ferric oxide (E172) (NF).

TABLE 17B
Composition of 60 mg dosage strength tablets containing Compound 1 (Formula E)

| | | | | |
|---|---|---|---|---|
| | Unit quantity | Percentage | | Reference to |
| Components | (mg/tablet) | (w/w) | Function | standards^{a} |

Intragranular

| | | | | |
|---|---|---|---|---|
| Compound 1 | 60.0 | 15.0 | Drug | In-house |
| | | | substance | monograph |
| Lactose monohydrate | 140.0 | 35.0 | Diluent | Ph. Eur.-NF |
| Microcrystalline cellulose 50 μm | 94.0 | 23.5 | Diluent | Ph. Eur.-NF |
| Hypromellose 6 mPa · s | 8.0 | 2.0 | Binder | Ph. Eur.-USP |
| Crospovidone type A | 6.0 | 1.5 | Disintegrant | Ph. Eur.-NF |
| Purified water^{b} | q.s. | — | Granulation | Ph. Eur.-USP |
| | | | solvent | |

Extragranular

| | | | | |
|---|---|---|---|---|
| Microcrystalline cellulose 90 μm | 84.0 | 21.0 | Diluent | Ph. Eur.-NF |
| Crospovidone type A | 6.0 | 1.5 | Disintegrant | Ph. Eur.-NF |
| Magnesium stearate^{c} | 2.0 | 0.5 | Lubricant | Ph. Eur.-NF |
| Core tablet mass | 400.0 | 100.0 | | |

Film-coating

| | | | | |
|---|---|---|---|---|
| Ready to use orange film-coating | 12.0 | 3.0 | Film-coating | In-house |
| agent^{d} | | | agent | monograph |
| Purified water^{b} | q.s | q.s. | Film-coating | Ph. Eur.-USP |
| | | | solvent | |
| Film-coated tablet mass | 412.0 | — | | |

^{a}When it is referred to a Pharmacopoeia, this means that the current edition of this Pharmacopoeia is applied.
^{b}Removed during manufacture.
^{c}From vegetable origin.
^{d}Aquarius ™ Prime Orange BAP313104 used. Aquarius ™ Prime Orange BAP313104 is composed of 62.50% hypromellose 2910 (Ph. Eur.-USP), 19.45% titanium dioxide (E171) [CI 77891] (Ph. Eur.-USP), 12.50% macrogol - polyethylene glycol (Ph. Eur.-USP), 4.32% yellow ferric oxide (E172) [CI 77492] (NF), and 1.23% red ferric oxide (E172) [CI 77491] (NF).

TABLE 17C
Composition of 120 mg dosage strength tablets containing Compound 1 (Formula E)

| | | | | |
|---|---|---|---|---|
| | Unit quantity | Percentage | | Reference to |
| Components | (mg/tablet) | (w/w) | Function | standards^{a} |

Intragranular

| | | | | |
|---|---|---|---|---|
| Compound 1 | 120.0 | 15.0 | Drug | In-house |
| | | | substance | monograph |
| Lactose monohydrate | 280.0 | 35.0 | Diluent | Ph. Eur.-NF |
| Microcrystalline cellulose 50 μm | 188.0 | 23.5 | Diluent | Ph. Eur.-NF |
| Hypromellose 6 mPa · s | 16.0 | 2.0 | Binder | Ph. Eur.-USP |
| Crospovidone type A | 12.0 | 1.5 | Disintegrant | Ph. Eur.-NF |
| Purified water^{b} | q.s. | — | Granulation | Ph. Eur.-USP |
| | | | solvent | |

Extragranular

| | | | | |
|---|---|---|---|---|
| Microcrystalline cellulose 90 μm | 168.0 | 21.0 | Diluent | Ph. Eur.-NF |
| Crospovidone type A | 12.0 | 1.5 | Disintegrant | Ph. Eur.-NF |
| Magnesium stearate^{c} | 4.0 | 0.5 | Lubricant | Ph. Eur.-NF |
| Core tablet mass | 800.0 | 100.0 | | |

Film-coating

| | | | | |
|---|---|---|---|---|
| Ready to use green film-coating | 24.0 | 3.0 | Film-coating | In-house |
| agent^{d} | | | agent | monograph |
| Purified water^{b} | q.s | q.s. | Film-coating | Ph. Eur.-USP |
| | | | solvent | |
| Film-coated tablet mass | 824.0 | — | | |

^{a}When it is referred to a Pharmacopoeia, this means that the current edition of this Pharmacopoeia is applied.
^{b}Removed during manufacture.
^{c}From vegetable origin.
^{d}Aquarius ™ Preferred HSP BPP316076 Green used. Aquarius ™ Preferred HSP BPP316076 Green is composed of 34.50% hypromellose 2910 (Ph. Eur.-USP), 28.00% Copovidone NF (Ph. Eur.) 18.36% titanium dioxide (E171) (Ph. Eur.-USP), 9.50% polyethylene glycol (Ph. Eur.-USP), 3.48% yellow iron oxide (E172) (NF), 3.16% FD&C Blue No. 2 Aluminum Lake Indigo carmine E132, and 3.00% Caprylic/Capric Triglyceride NF (Ph. Eur.).

### Example 3: Small-Scale Film-Coating Tests of 10 mg and 60 mg Dosages

The film-coating was carried out according to the procedure of Example 2 and with the process parameters described in Table 18 on Formula E Batch 2.

TABLE 18
Characteristics of 10 mg film-coated tablets of Formula E Batch 2

| | | Formula E |
|---|---|---|
| Characteristics | Target limits | Batch 2 |
| Average mass (mg) | 69 mg ± 5 mg | 69.6 ± 1 |
| Hardness | For information | 76 |
| (N) (n = 10) | [min-max] | [62-93] |
| Thickness | Mean | 2.51 |
| (mm) (n = 10) | [min-max] | [2.47-2.56] |
| Disaggregation | <15 | 3 min 56 |
| (max duration, min) | | |

TABLE 19
Characteristics of 60 mg film-coated tablets of Formula E Batch 2

| | | Formula E |
|---|---|---|
| Characteristics | Target limits | Bacth 2 |
| Average mass (mg) | 412 mg ± 21 mg | 405.1 ± 4.2 mg |
| Hardness | For information mean | 282 |
| (N) (n = 10) | [min-max] | [264-300] |
| Thickness | Mean | 5.65 |
| (mm) (n = 10) | [min-max] | [5.63-5.69] |
| Disaggregation | <15 | 3 min 45 |
| (max duration, min) | | |
| Moisture content (%, KF) | | 3.9 |
| Uniformity of content | Pi | 97.25% CV: 0.85% |

The film-coated tablets of both batches have suitable galenic characteristics as well as good quality of film-coating taking into consideration the homogeneity of the color, smooth appearance and the absence of defects. Between the compression step and the film-coating step, there was an increase in hardness and disintegration time for both dosages 10 mg and 60 mg.

An investigation was carried out by characterizing these batches of TO film-coated tablets and then every day for three days: the tablets were stored in open vials in the compression room at ambient temperature and humidity. It appeared that a decrease is observed over time for the values of hardness and disintegration times to reach a plateau for both dosages.

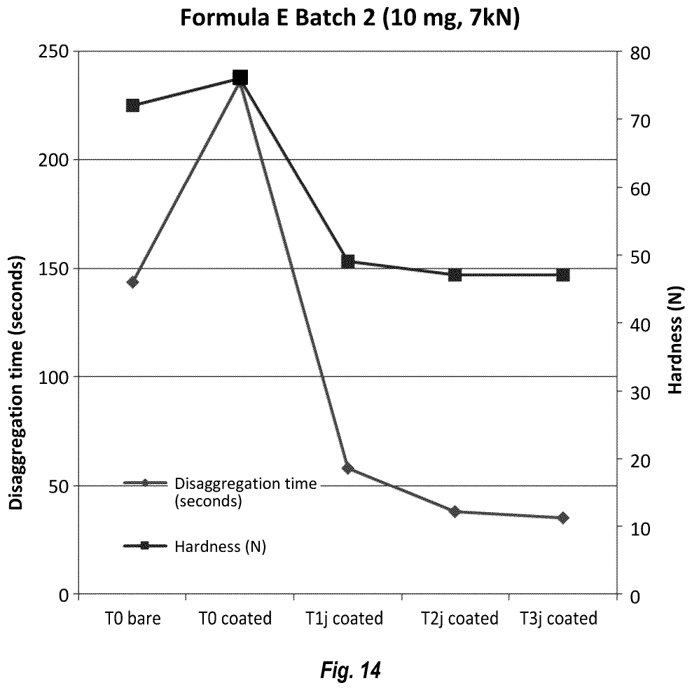

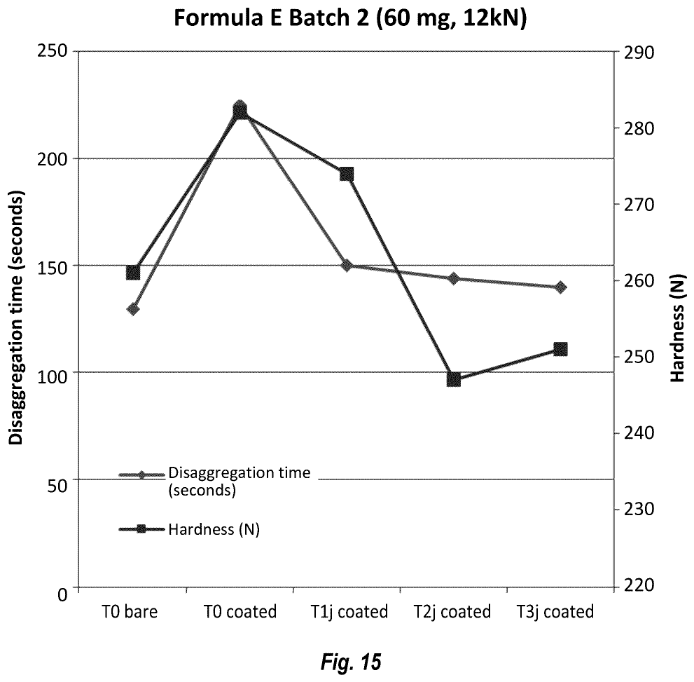

FIGS. 14 and 15 show the hardness curves and disaggregation time for the 10 mg and 60 mg dosage strength tablets of Formula E Batch 2.

### Example 4: Dissolution of 10 mg and 60 mg Dosage Strength Tablets Containing Compound 1

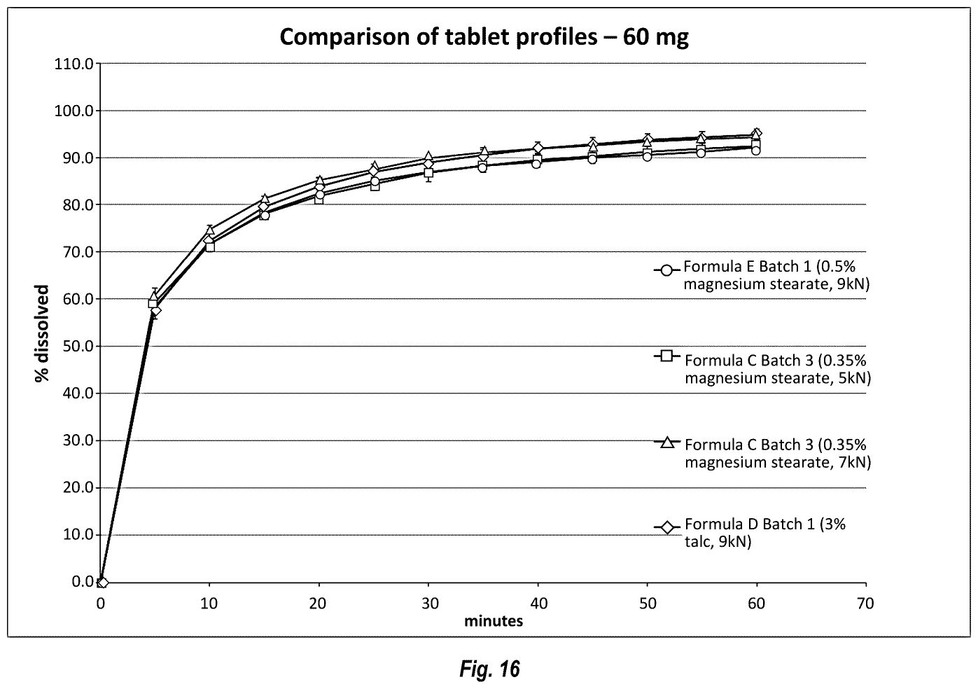

As shown in FIG. 16, the dissolution profiles for the 60 mg dosage strength tablets are comparable for tablets of Formula E Batch 1, Formula C Batch 3, Formula D Batch 1, and Formula E Batch 2. The addition of talc and amount of magnesium stearate had no impact on dissolution, and 80% release was reached before 20 minutes.

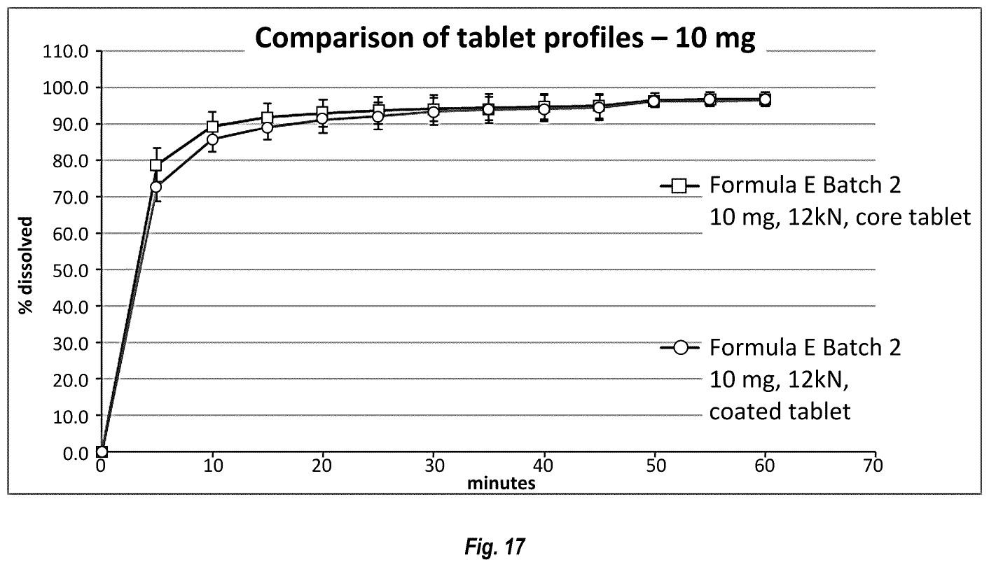

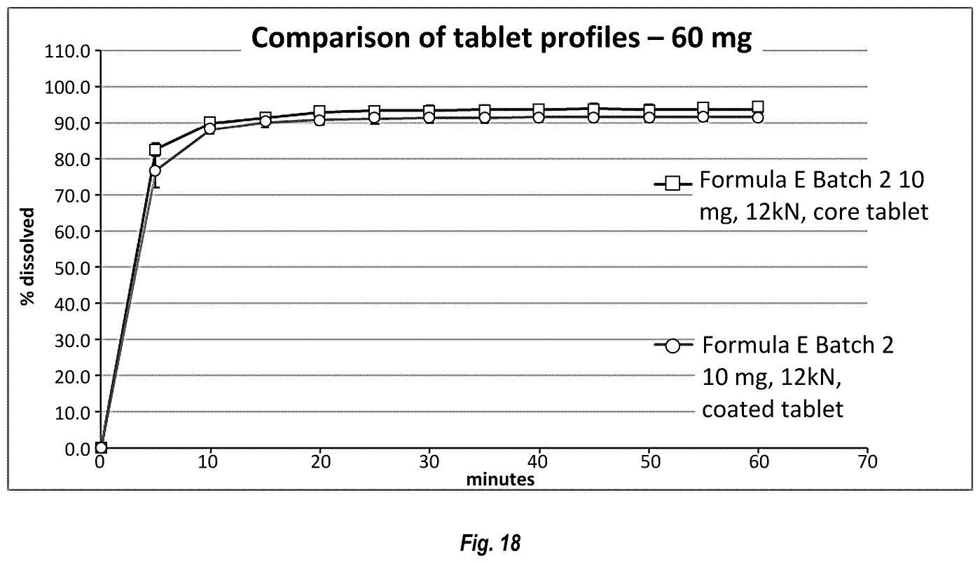

The dissolution of 10 mg and 60 mg dosage strength tablets of Formula E Batch 1, are shown in FIGS. 17 and 18.

The comparison of the profiles of the core tablets versus film-coated tablets shows the similarity of the profiles for the orange Opadry® QX film-coating agent regardless of the dosage of the tablet, 10 or 60 mg.

The present disclosure includes, for example, any one or a combination of the following embodiments:

Embodiment 1: A tablet comprising: at least one compound chosen from (R)-1-(1-acryloylpiperidin-3-yl)-4-amino-3-(4-phenoxyphenyl)-1H-imidazo[4,5-c]pyridin-2(3H)-one (Compound 1) and pharmaceutically acceptable salts thereof; at least one diluent; at least one binder; at least one disintegrant; and at least one lubricant.

Embodiment 2: The tablet of embodiment 1, further comprising at least one flow agent.

Embodiment 3: The tablet of any one of the preceding embodiments, wherein the tablet comprises an intragranular core and an extragranular portion.

Embodiment 4: The tablet of embodiment 3, wherein the at least one diluent is chosen from lactose monohydrate and microcrystalline cellulose.

Embodiment 5: The tablet of embodiment 4, wherein the at least one diluent is present in a total amount ranging from about 70% to about 90% by weight.

Embodiment 6: The tablet of embodiment 5, wherein the at least one diluent is present in the intragranular core in a total amount ranging from about 50% to about 70% by weight.

Embodiment 7: The tablet of embodiment 5 or 6, wherein the at least one diluent is present in the extragranular portion in a total amount ranging from about 10% to about 30% by weight.

Embodiment 8: The tablet of any one of the preceding embodiments, wherein the at least one binder is hydroxypropyl methylcellulose (hypromellose).

Embodiment 9: The tablet of embodiment 8, wherein the at least one binder is present in a total amount ranging from about 0.1% to about 5% by weight.

Embodiment 10: The tablet of embodiment 3, wherein the at least one disintegrant is cross linked polyvinyl N-pyrrolidone (crospovidone). Embodiment 11: The tablet of embodiment 10, wherein the at least one disintegrant is present in a total amount ranging from about 0.1% to about 5% by weight.

Embodiment 12: The tablet of embodiment 11, wherein the at least one disintegrant is present in the intragranular core in a total amount ranging from about 0.5% to about 3% by weight.

Embodiment 13: The tablet of embodiment 11 or 12, wherein the at least one disintegrant is present in the extragranular portion in a total amount ranging from about 0.5% to about 3% by weight.

Embodiment 14: The tablet of any one of the preceding embodiments, wherein the at least one lubricant is magnesium stearate.

Embodiment 15: The tablet of embodiment 14, wherein the at least one lubricant is present in a total amount ranging from about 0.1% to about 5% by weight.

Embodiment 16: The tablet of embodiment 2, wherein the at least one flow agent is talc.

Embodiment 17: The tablet of embodiment 16, wherein the at least one flow agent is present in a total amount ranging from about 1% to about 5% by weight.

Embodiment 18: The tablet of embodiment 3, wherein the intragranular core comprises at least one diluent, at least one binder, and at least one disintegrant.

Embodiment 19: The tablet of embodiment 3 or 18, wherein the extragranular portion comprises at least one diluent, at least one disintegrant, and at least one lubricant.

Embodiment 20: The tablet of any one of the preceding embodiments, wherein Compound 1 is present in at least 50% crystalline form, such as at least 60%, at least 70%, at least 80%, at least 90%, at least 95%, at least 96%, at least 97%, at least 98%, at least 99%, 99.5%, or 100% crystalline.

Embodiment 21: The tablet of any one of the preceding embodiments, wherein Compound 1 is present in an amount ranging from about 0.9 mg to about 125 mg.

Embodiment 22: The tablet of any one of the preceding embodiments, wherein Compound 1 is present in an amount of about 1 mg, about 2.5 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, and about 125 mg.

Embodiment 23: The tablet of any one of the preceding embodiments, wherein Compound 1 is present in an amount of about 10 mg.

Embodiment 24: The tablet of any one of the preceding embodiments, wherein Compound 1 is present in an amount of about 60 mg.

Embodiment 25: The tablet of any one of the preceding embodiments, wherein Compound 1 is present in an amount of about 120 mg.

Embodiment 26: The tablet of any one of the preceding embodiments, wherein Compound 1 is present in an amount of about 5% to 25% by weight.

Embodiment 27: The tablet of any one of the preceding embodiments, further comprising at least one coating layer.

Embodiment 28: The tablet of embodiment 27, wherein the coating layer comprises macrogol polyvinyl alcohol grafted copolymer, optionally further comprising talc with titanium dioxide.

Embodiment 29: The tablet of embodiment 27, wherein the coating layer comprises hydroxypropyl methylcellulose.

Embodiment 30: The tablet of embodiment 29, wherein the coating layer further comprises at least one of titanium dioxide and macrogol polyethylene glycol.

Embodiment 31: The tablet of embodiment 29, wherein the coating layer further comprises at least one of copovidone and titanium dioxide.

Embodiment 32: The tablet of embodiment 1, wherein the tablet comprises: about 15% to about 45% by weight lactose monohydrate, about 35% to about 60% by weight microcrystalline cellulose, about 1% to about 5% by weight hydroxypropyl methylcellulose, about 1% to about 5% by weight crospovidone, and about 0.1% to about 3% by weight magnesium stearate.

Embodiment 33: The tablet of embodiment 32, wherein the tablet comprises an intragranular core and an extragranular portion.

Embodiment 34: The tablet of embodiment 33, wherein the intragranular core comprises about 20% to about 40% by weight microcrystalline cellulose.

Embodiment 35: The tablet of embodiment 33, wherein the extragranular portion comprises about 15% to about 25% by weight microcrystalline cellulose.

Embodiment 36: The tablet of embodiment 33, wherein the intragranular core comprises about 0.5% to about 2.5% by weight crospovidone.

Embodiment 37: The tablet of embodiment 33, wherein the extragranular portion comprises about 0.5% to about 2.5% by weight crospovidone.

Embodiment 38: The tablet of any one of embodiments 32-37, wherein Compound 1 is present in an amount of about 5% to 25% by weight.

Embodiment 39: The tablet of embodiment 1, wherein the tablet comprises: about 30% to about 40% by weight lactose monohydrate, about 40% to about 50% by weight microcrystalline cellulose, about 1% to about 3% by weight hydroxypropyl methylcellulose, about 2% to about 4% by weight crospovidone, and about 0.1% to about 1% by weight magnesium stearate.

Embodiment 40: The tablet of embodiment 39, wherein the tablet comprises an intragranular core and an extragranular portion.

Embodiment 41: The tablet of embodiment 40, wherein the intragranular core comprises about 20% to about 30% by weight microcrystalline cellulose.

Embodiment 42: The tablet of embodiment 40, wherein the extragranular portion comprises about 15% to about 25% by weight microcrystalline cellulose.

Embodiment 43: The tablet of embodiment 40, wherein the intragranular core comprises about 0.5% to about 2.5% by weight crospovidone.

Embodiment 44: The tablet of embodiment 40, wherein the extragranular portion comprises about 0.5% to about 2.5% by weight crospovidone.

Embodiment 45: The tablet of any one of embodiments 39-44, wherein Compound 1 is present in an amount of about 10% to 20% by weight.

Embodiment 46: The tablet of embodiment 1, wherein the tablet comprises: about 35.0% by weight lactose monohydrate, about 44.5% by weight microcrystalline cellulose, about 2.0% by weight hydroxypropyl methylcellulose, about 3.0% by weight crospovidone, and about 0.5% by weight magnesium stearate.

Embodiment 47: The tablet of embodiment 46, wherein the tablet comprises an intragranular core and an extragranular portion.

Embodiment 48: The tablet of embodiment 47, wherein the intragranular core comprises 23.5% by weight microcrystalline cellulose.

Embodiment 49: The tablet of embodiment 47, wherein the extragranular portion comprises 21.0% by weight microcrystalline cellulose.

Embodiment 50: The tablet of embodiment 47, wherein the intragranular core comprises about 1.5% by weight crospovidone.

Embodiment 51: The tablet of embodiment 47, wherein the extragranular portion comprises about 1.5% by weight crospovidone.

Embodiment 52: The tablet of any one of embodiments 46-51, wherein Compound 1 is present in an amount of about 15% by weight.

Embodiment 53: The tablet of embodiment 1, wherein the tablet comprises: about 28.8% by weight lactose monohydrate, about 55.8% by weight microcrystalline cellulose, about 2.0% by weight hydroxypropyl methylcellulose, about 3.0% by weight crospovidone, and about 0.4% by weight magnesium stearate.

Embodiment 54: The tablet of embodiment 53, wherein the tablet comprises an intragranular core and an extragranular portion.

Embodiment 55: The tablet of embodiment 54, wherein the intragranular core comprises 34.8% by weight microcrystalline cellulose.

Embodiment 56: The tablet of embodiment 54, wherein the extragranular portion comprises 21.0% by weight microcrystalline cellulose.

Embodiment 57: The tablet of embodiment 54, wherein the intragranular core comprises about 1.5% by weight crospovidone.

Embodiment 58: The tablet of embodiment 54, wherein the extragranular portion comprises about 1.5% by weight crospovidone.

Embodiment 59: The tablet of any one of embodiments 53-58, wherein Compound 1 is present in an amount of about 10% by weight.

Embodiment 60: The tablet of embodiment 1, wherein the tablet comprises: about 23.5% by weight lactose monohydrate, about 56.2% by weight microcrystalline cellulose, about 2.0% by weight hydroxypropyl methylcellulose, about 3.0% by weight crospovidone, and about 0.4% by weight magnesium stearate.

Embodiment 61: The tablet of embodiment 60, wherein the tablet comprises an intragranular core and an extragranular portion.

Embodiment 62: The tablet of embodiment 61, wherein the intragranular core comprises 35.2% by weight microcrystalline cellulose.

Embodiment 63: The tablet of embodiment 61, wherein the extragranular portion comprises 21.0% by weight microcrystalline cellulose.

Embodiment 64: The tablet of embodiment 61, wherein the intragranular core comprises about 1.5% by weight crospovidone.

Embodiment 65: The tablet of embodiment 61, wherein the extragranular portion comprises about 1.5% by weight crospovidone.

Embodiment 66: The tablet of any one of embodiments 60-65, wherein Compound 1 is present in an amount of about 15% by weight.

Embodiment 67: The tablet of embodiment 1, wherein the tablet comprises: about 35.2% by weight lactose monohydrate, about 44.5% by weight microcrystalline cellulose, about 2.0% by weight hydroxypropyl methylcellulose, about 3.0% by weight crospovidone, and about 0.4% by weight magnesium stearate.

Embodiment 68: The tablet of embodiment 67, wherein the tablet comprises an intragranular core and an extragranular portion.

Embodiment 69: The tablet of embodiment 68, wherein the intragranular core comprises 23.5% by weight microcrystalline cellulose.

Embodiment 70: The tablet of embodiment 68, wherein the extragranular portion comprises 21.0% by weight microcrystalline cellulose.

Embodiment 71: The tablet of embodiment 68, wherein the intragranular core comprises about 1.5% by weight crospovidone.

Embodiment 72: The tablet of embodiment 68, wherein the extragranular portion comprises about 1.5% by weight crospovidone.

Embodiment 73: The tablet of any one of embodiments 67-72, wherein Compound 1 is present in an amount of about 15% by weight.

Embodiment 74: The tablet of embodiment 2, wherein the tablet comprises: about 32.2% by weight lactose monohydrate, about 44.5% by weight microcrystalline cellulose, about 2.0% by weight hydroxypropyl methylcellulose, about 3.0% by weight crospovidone, about 0.4% by weight magnesium stearate; and about 3.0% by weight talc.

Embodiment 75: The tablet of embodiment 74, wherein the tablet comprises an intragranular core and an extragranular portion.

Embodiment 76: The tablet of embodiment 75, wherein the intragranular core comprises 23.5% by weight microcrystalline cellulose.

Embodiment 77: The tablet of embodiment 75, wherein the extragranular portion comprises 21.0% by weight microcrystalline cellulose.

Embodiment 78: The tablet of embodiment 75, wherein the intragranular core comprises about 1.5% by weight crospovidone.

Embodiment 79: The tablet of embodiment 75, wherein the extragranular portion comprises about 1.5% by weight crospovidone.

Embodiment 80: The tablet of any one of the preceding embodiments, wherein Compound 1 is present in an amount of about 15% by weight.

Embodiment 81: The tablet of any one of embodiments 32-80, further comprising a film-coating agent.

Embodiment 82: The tablet of embodiment 81, wherein the film coating is present in an amount of about 2% to about 5% relative to the total weight of the tablet.

Embodiment 83: The tablet of embodiment 82, wherein the film coating is present in an amount of about 3% relative to the total weight of the tablet.

Embodiment 84: The tablet of any one of embodiments 53-66, wherein the ratio of lactose to intragranular microcrystalline cellulose is about 40:60 by weight.

Embodiment 85: The tablet of any one of embodiments 46-52 and 67-79, wherein the ratio of lactose to intragranular microcrystalline cellulose is about 60:40 by weight.

Embodiment 86: The tablet of embodiment 27, wherein the coating layer comprises macrogol polyvinyl alcohol grafted copolymer, talc, titanium dioxide, glycerol monocaprylocaprate type 1/mono/diglycerides/glycerol of fatty acids, yellow ferric oxide, polyvinyl alcohol partly hydrolyzed, and red ferric oxide.

Embodiment 87: The tablet of embodiment 27, wherein the coating layer comprises hydroxypropyl methylcellulose, titanium dioxide, macrogol polyethylene glycol, yellow ferric oxide, and red ferric oxide.

Embodiment 88: The tablet of embodiment 27, wherein the coating layer comprises hydroxypropyl methylcellulose, copovidone, titanium dioxide, polyethylene glycol, yellow ferric oxide, indigo carmine aluminum lake, and caprylic/capric triglyceride.

Embodiment 89: A method of treating a disease or condition mediated by BTK in a patient in need thereof, comprising administering to the patient the tablet of any one of embodiments 1 to 88.

Embodiment 90: Use of the tablet of any one of embodiments 1 to 88 for treating a disease involving mediation of the BTK receptor.

1. A tablet comprising:
at least one compound chosen from (R)-1-(1-acryloylpiperidin-3-yl)-4-amino-3-(4-phenoxyphenyl)-1H-imidazo[4,5-c]pyridin-2(3H)-one (Compound 1) and pharmaceutically acceptable salts thereof;
at least one diluent;
at least one binder;
at least one disintegrant; and
at least one lubricant.

2. The tablet of claim 1, further comprising at least one flow agent.

3. The tablet of claim 1, wherein the tablet comprises an intragranular core and an extragranular portion.

4. The tablet of claim 3, wherein the at least one diluent is chosen from lactose monohydrate and microcrystalline cellulose.

5. The tablet of claim 4, wherein the at least one diluent is present in a total amount ranging from about 70% to about 90% by weight.

6. The tablet of claim 5, wherein the at least one diluent is present in the intragranular core in a total amount ranging from about 50% to about 70% by weight.

7. The tablet of claim 5, wherein the at least one diluent is present in the extragranular portion in a total amount ranging from about 10% to about 30% by weight.

8. The tablet of claim 1, wherein the at least one binder is hydroxypropyl methylcellulose (hypromellose).

9. The tablet of claim 8, wherein the at least one binder is present in a total amount ranging from about 0.1% to about 5% by weight.

10. The tablet of claim 3, wherein the at least one disintegrant is cross linked polyvinyl N-pyrrolidone (crospovidone).

11. The tablet of claim 10, wherein the at least one disintegrant is present in a total amount ranging from about 0.1% to about 5% by weight.

12. The tablet of claim 11, wherein the at least one disintegrant is present in the intragranular core in a total amount ranging from about 0.5% to about 3% by weight.

13. The tablet of claim 11, wherein the at least one disintegrant is present in the extragranular portion in a total amount ranging from about 0.5% to about 3% by weight.

14. The tablet of claim 1, wherein the at least one lubricant is magnesium stearate.

15. The tablet of claim 14, wherein the at least one lubricant is present in a total amount ranging from about 0.1% to about 5% by weight.

16. The tablet of claim 2, wherein the at least one flow agent is talc.

17. The tablet of claim 16, wherein the at least one flow agent is present in a total amount ranging from about 1% to about 5% by weight.

18. The tablet of claim 3, wherein the intragranular core comprises at least one diluent, at least one binder, and at least one disintegrant.

19. The tablet of claim 3, wherein the extragranular portion comprises at least one diluent, at least one disintegrant, and at least one lubricant.

20. The tablet of claim 1, wherein Compound 1 is present in at least 50% crystalline form.

21. (canceled)

22. (canceled)

23. (canceled)

24. The tablet of claim 1, wherein Compound 1 is present in an amount of about 60 mg.

25. The tablet of claim 1, wherein Compound 1 is present in an amount of about 120 mg.

26. (canceled)

27. (canceled)

28. (canceled)

29. (canceled)

30. (canceled)

31. (canceled)

32. The tablet of claim 1, wherein the tablet comprises:
about 15% to about 45% by weight lactose monohydrate,
about 35% to about 60% by weight microcrystalline cellulose,
about 1% to about 5% by weight hydroxypropyl methylcellulose,
about 1% to about 5% by weight crospovidone, and
about 0.1% to about 3% by weight magnesium stearate.

33. The tablet of claim 32, wherein the tablet comprises an intragranular core and an extragranular portion.

34-88. (canceled)

89. A method of treating a disease or condition mediated by BTK in a patient in need thereof, comprising administering to the patient the tablet of claim 1.

90. (canceled)
